SEQUENCE LISTING (3 sequences)

SEQ ID NO: 1 - DNA, 27 bp, Artificial
  Description of Artificial Sequence Primer
tcaaggtgcagttcattatcatcactg

SEQ ID NO: 2 - DNA, 27 bp, Artificial
  Description of Artificial Sequence Primer
cttcagttgcaggatcttcagctgacc

SEQ ID NO: 3 - DNA, 28 bp, Artificial
  Description of Artificial Sequence Primer
gctaaagcgcatgctccagactgccttg

(19) United States
(12) Patent Application Publication
(10) Pub. No.: US 2004/0115224 A1
(43) Pub. Date: Jun. 17, 2004

(54) Preparation and administration of hybrid cell vaccines for the prevention of cancer

(76) Inventor: Tsuneya Ohno, Boston, MA (US)

Correspondence Address: JONES DAY, 222 EAST 41ST ST, NEW YORK, NY, 10017, US

(21) Appl. No.: 10/320,779

(22) Filed: Dec. 16, 2002

(51) Int. Cl. A61K 39/00

(52) U.S. Cl. 424/277.1

(57) ABSTRACT

The present invention relates to methods for preventing cancer and for treating and preventing development and progression of pre-cancerous lesions by administering a therapeutically effective dose of fusion cells formed by fusion of dendritic cells and precancerous non-dendritic cells, either alone or in combination with a cytokine or other molecule which stimulates or induces a cytotoxic T cell response and/or a humoral immune response.

### 1. INTRODUCTION

The present invention relates to methods for preventing cancer and for treating pre-cancerous lesions by administering a therapeutically effective dose of fusion cells formed by fusion of dendritic cells and pre-cancerous non-dendritic cells, and in certain embodiments, administering such fusion cells in combination with a cytokine or other molecule that stimulates a cytotoxic T cell (CTL) response and/or a humoral immune response.

### 2. BACKGROUND OF THE INVENTION

There is great interest in the development of an effective immunotherapeutic composition for preventing cancer. Success at such an immunotherapeutic approach will require the development of a composition that is both capable of eliciting a very strong immune response, that is extremely specific for the target tumor or infected cell.

2.1 The Immune Response

Cells of the immune system arise from pluripotent stem cells through two main lines of differentiation, the lymphoid lineage and the myeloid lineage. The lymphoid lineage produces lymphocytes, such as T cells, B cells, and natural killer cells, while the myeloid lineage produces monocytes, macrophages, and neutrophils and other accessory cells, such as dendritic cells, platelets, and mast cells. There are two main types of T cells of the lymphoid lineage, cytotoxic T lymphocytes (“CTLs”) and helper T cells which mature and undergo selection in the thymus, that are distinguished by the presence of one of two surface markers, CD8 (CTLs) or CD4 (helper T cells).

Lymphocytes circulate and search for invading foreign pathogens and antigens that tend to become trapped in secondary lymphoid organs, such as the spleen and the lymph nodes. Antigens are taken up in the periphery by the antigen-presenting cells (APCs) that migrate to secondary lymphoid organs. Interaction between T cells and APCs triggers several effector pathways, including activation of B cells and antibody production, activation of CD8⁺ cytotoxic T lymphocytes (CD8⁺ CTLs), and stimulation of cytokine production by T cells.

Activation of naive B cells, to produce antibodies, requires two signals: (1) recognition and binding of specific antigens by surface-bound receptors (B cell receptors, or “BCR”), which then cluster together along with BCR-associated signaling molecules, and (2) a co-stimulatory signal provided by binding of the CD40 receptor on the B cell surface by the CD40L ligand carried on the surface of activated T-helper cells (Th). Activated B cells undergo clonal expansion, somatic hypermutation, affinity maturation, and isotype switching, in which the heavy chain class of the secreted antibody is established. Selection of the antibody heavy-chain class, in turn, is determined by the collection of cytokines contacting the B cell at the time isotype switching is carried out.

The heavy-chain constant region (Fc) of an antibody influences the function of that antibody in vivo. For example, the Fc portion of the IgG class of antibodies is recognized and bound by cell-surface receptors of professional phagocytic cells such as macrophage and neutrophils, thereby facilitating ingestion and destruction of IgG-bound antigens and/or cells opsonized in this manner. In addition, clusters of IgG antibodies bound, e.g., to multiple copies of a cell-surface antigen will fix and activate the complement system, leading to the destruction of that cell.

In contrast to antigen recognition and binding by BCR and antibodies, T cells require that antigenic proteins be processed by one of two distinct routes, depending upon whether the origin of the antigen is intracellular or extracellular, and presented as part of a cell-surface-bound complex. Intracellular or endogenous protein antigens are presented to CD8⁺ CTLs by class I major histocompatibility complex (MHC) molecules that are expressed in most cell types, including tumor cells. Extracellular antigenic determinants are presented on the cell surface of “specialized” or “professional” APCs, such as dendritic cells and macrophages, as class II MHC molecules-antigen complexes that are recognized by CD4⁺ “helper” T cells (see generally, W. E. Paul, ed., Fundamental Immunology. New York: Raven Press, 1984).

Class I and class II MHC molecules are the most polymorphic proteins known. A further degree of heterogeneity of MHC molecules is generated by the combination of class I and class II MHC molecules, known as the MHC haplotype. In humans, HLA-A, HLA-B and HLA-C, three distinct genetic loci located on a single chromosome, encode class I molecules. Because T cell receptors specifically bind complexes comprising an antigenic peptide and the polymorphic portion of an MHC molecule, T cells respond poorly when an MHC molecule of a different genetic type is encountered. This specificity results in the phenomenon of MHC-restricted T cell recognition and T cell cytotoxicity.

Lymphocytes circulate in the periphery and become “primed” in the lymphoid organs on encountering the appropriate signals (Bretscher and Cohn, 1970, Science 169:1042-1049). The first signal is received through the T cell receptor after it engages antigenic peptides displayed by class I MHC molecules on the surface of APCs. The second signal is provided either by a secreted chemical signal or cytokine, such as interleukin-1 (IL-1), interferon-γ, interleukin-2 (IL-2), interleukin-4 (IL-4), interleukin-7 (IL-7), and interleukin-12 (IL-12), produced by CD4⁺ helper T cells or dendritic cells, or by a plasma-membrane-bound co-stimulatory molecule, such as B7 (a term which includes B7.1 and B7.2 molecules), which is present on the antigen-presenting-cell membrane and is recognized by a co-receptor on the cell surface of helper T cells, called CD28, a member of the Ig superfamily. Interferon-y and IL-12 production are associated with the helper T cell subtype known as TH₁ that promote development of CD8⁺ T cells, and IL-4 production, which is associated with the T helper cell subtype known as TH₂ that promotes development and activation of antibody-producing B cells.

In addition to antigen-specific interactions during antigen presentation, antigen non-specific adhesive that stabilize binding of T lymphocytes to APC stimulation. More specifically, receptor molecules on APC, such as ICAM-1/CD54, LFA-3/CD58, and B7, bind corresponding co-receptors on T cells. Helper T cells receiving both signals are activated to proliferate and to secrete a variety of interleukins. CTLs receiving both signals are activated to kill target cells that carry the same class I MHC molecule and the same antigen that originally induced CTL activation. Accordingly, CD8⁺ CTLs are important in resisting cancer and pathogens, as well as rejecting allografts (Terstappen et al., 1992, Blood 79:666-677). However, T cells receiving the first signal in the absence of co-stimulation become anergized, leading to tolerance (Lamb et al., 1983, J. Exp. Med. 157:1434-1447; Mueller et al., 1989, Annu. Rev. Immunol. 7:445-480; Schwartz, 1992, Cell 71:1065-1068; Mueller and Jenkins, 1995, Curr. Opin. Immunol. 7:375-381).

2.2 Pathobiology of Cancer

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor pre-neoplastic changes, which may under certain conditions progress to neoplasia. Therefore, during the progression of this multistep process, pre-cancerous cells accumulate that comprise at least one genetic allele that distinguishes a pre-cancerous cell from a normal cell. Such genetic differences can result in the expression of tumor-specific antigens, over-expression of normal cellular proteins, and/or altered cellular distribution of normal and/or tumor-specific antigens. In certain instances, these alterations may result in cell-surface expression of an altered cell-surface protein or of a normal protein that is generally not transported to the cell surface.

Accumulation of pre-cancerous cells is detected as pre-malignant abnormal cell growth that is exemplified by hyperplasia, metaplasia, or most particularly, dysplasia (for a review of such abnormal growth conditions, see Robbins and Angell, 1976, Basic Pathology, 2d. Ed., W. B. Saunders Co., Philadelphia, pp. 68-79). Hyperplasia is a form of controlled cell proliferation involving an increase in cell number in a tissue or organ, without significant alteration in structure or function. One example of hyperplasia is endometrial hyperplasia, which often precedes endometrial cancer. Metaplasia is a form of controlled cell growth in which one type of adult cell or fully-differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic growth involving a loss individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

The neoplastic lesion, which comprises the pre-cancerous and cancerous cells described above, may evolve clonally as pre-cancerous cells accumulation a plurality of genetic alterations that provide an increasing capacity for invasion, growth, metastasis, and heterogeneity, especially under conditions in which the neoplastic cell escapes the host's immune surveillance (Roitt et al, 1993, Immunology, 3^{rd} Ed., Mosby, St. Louis, pps. 17.1-17.12).

2.3 Immunotherapy Against Cancer

The cytotoxic T cell response is a very important host response for the control of growth of antigenic tumor cells (Anichimi et al., 1987, Immunol. Today 8:385-389). Studies with experimental animal tumors as well as spontaneous human tumors have demonstrated that many tumors express antigens that can induce an immune response. Some antigens are unique to the tumor, and some are found on both tumor and normal cells. Several factors influence the immunogenicity of the tumor, including, for example, the specific type of carcinogen involved, and immunocompetence of the host and the latency period (Old et al., 1962, Ann. N.Y. Acad. Sci. 101:80-106; Bartlett, 1972, J. Natl. Cancer. Inst. 49:493-504). It has been demonstrated that T cell-mediated immunity is of critical importance for rejection of virally and chemically induced tumors (Klein et al., 1960, Cancer Res. 20:1561-1572; Tevethia et al., 1974, J. Immunol. 13:1417-1423).

Adoptive immunotherapy for tumors refers to the therapeutic approach wherein immune cells with antitumor activity are administered to a tumor-bearing host, with the objective that the cells cause regression of an established tumor, either directly or indirectly. Immunization of hosts bearing established tumors with tumor cells or tumor antigens, as well a spontaneous tumors, has often been ineffective since the tumor may have already elicited an immunosuppressive response (Greenberg, 1987, Chapter 14, in Basic and Clinical Immunology, 6th ed., ed. by Stites, Stobo and Wells, Appleton and Lange, pp. 186-196; Bruggen, 1993). Thus, prior to immunotherapy, it had been necessary to reduce the tumor mass and deplete all the T cells in the tumor-bearing host (Greenberg et al., 1983, page 301-335, in “Basic and Clinical Tumor Immunology”, ed. Herbermann R R, Martinus Nijhoff).

Animal models have been developed in which hosts bearing advanced tumors can be treated by the transfer of tumor-specific syngeneic T cells (Mulé et al., 1984, Science 225:1487-1489). Investigators at the National Cancer Institute (NCI) have used autologous reinfusion of peripheral blood lymphocytes or tumor-infiltrating lymphocytes (TIL), T cell cultures from biopsies of subcutaneous lymph nodules, to treat several human cancers (Rosenberg, S. A., U.S. Pat. No. 4,690,914, issued Sep. 1, 1987; Rosenberg et al., 1988, N. Engl. J. Med., 319:1676-1680). For example, TIL expanded in vitro in the presence of IL-2 have been adoptively transferred to cancer patients, resulting in tumor regression in select patients with metastatic melanoma. Melanoma TIL grown in IL-2 have been identified as CD3⁺-activated T lymphocytes, which are predominantly CD8⁺ cells with unique in vitro anti-tumor properties. Many long-term melanoma TIL cultures lyse autologous tumors in a specific class I MHC-antigen complex and T cell receptor-dependent manner (Topalian et al., 1989, J. Immunol. 142:3714).

Application of these methods for treatment of human cancers would entail isolating a specific set of tumor-reactive lymphocytes present in a patient, expanding these cells to large numbers in vitro, and then putting these cells back into the host by multiple infusions. Since T cells expanded in the presence of IL-2 are dependent upon IL-2 for survival, infusion of IL-2 after cell transfer prolongs the survival and augments the therapeutic efficacy of cultured T cells (Rosenberg et al., 1987, N. Engl. J. Med. 316:889-897). However, the toxicity of the high-dose IL-2 and activated lymphocyte treatment has been considerable, including high fevers, hypotension, damage to the endothelial wall due to capillary leak syndrome, and various adverse cardiac events such as arrhythmia and myocardial infarction (Rosenberg et al., 1988, N. Engl. J. Med. 319:1676-1680). Furthermore, the demanding technical expertise required to generate TILs, the quantity of material needed, and the severe adverse side effects limit the use of these techniques to specialized treatment centers.

CTLs specific for class I MHC-peptide complexes could be used in treatment or prevention of cancer, and ways have been sought to generate such CTLs in vitro without the requirement for priming in vivo. These include the use of dendritic cells pulsed with appropriate antigens (Inaba et al., 1987, J. Exp. Med. 166:182-194; Macatonia et al., 1989, J. Exp. Med. 169:1255-1264; De Bruijn et al., 1992, Eur. J. Immunol. 22:3013-3020). RMA-S cells (mutant cells expressing high numbers of “empty” cell surface class I MHC molecules) loaded with peptide (De Bruijn el al., 1991, Eur. J. Immunol. 21:2963-2970; De Bruijn et al., 1992, supra; Houbiers et al., 1993, Eur. J. Immunol. 26:2072-2077) and macrophage phagocytosed-peptide loaded beads (De Bruijn et al., 1995, Eur. J. Immunol. 25, 1274-1285).

Dendritic cells, which are potent antigen presenting cells, have recently been utilized as an adjuvant for cancer immunotherapy. Gong et al. reported that inoculation of dendritic cells fused with tumor cell induced anti-tumor immunity in mice (Gong et al., 1997, supra). Successful clinical application of fused with tumor cell has also been reported (Kugler et al., 2000, Nat Med 6, 332-336). Fusion of B cells or dendritic cells with tumor cells has been previously demonstrated to elicit anti-tumor immune responses in animal models (Guo et al., 1994, Science, 263:518-520; Stuhler and Walden, 1994, Cancer Immunol. Immuntother. 1994, 39:342-345; Gong et al., 1997, Nat. Med. 3:558-561; Celluzzi, 1998, J. Immunol. 160:3081-3085; Gong, PCT publication WO 98/46785, dated Oct. 23, 1998). In particular, immunization with hybrids of tumor cells and antigen presenting cells has been shown to result in protective immunity in various rodent models.

However, despite such progress, cancer cells have acquired various strategies to evade the host immunosurveillance, hampering the development of effective immunotherapy. The current treatments, while stimulating protective immunity, are not always successful at treating a patient who already has an established disease. In addition, such treatments are generally not effective for prevention of cancer in those patients who, although they may be tumor-free, nevertheless carry pre-cancerous lesions. Thus, a need exists for a therapeutic composition which can be used for prevention of neoplastic disease, prevent recurrence of neoplastic disease, and cause the regression of an existing tumor in a patient. Moreover, there is an especially acute need for such compositions for the prophylactic treatment of those patients known to carry one or more genetic markers or alleles that are strongly predictive of an eventual development of neoplastic disease.

Citation or discussion of a reference herein shall not be construed as an admission that such is prior art to the present invention.

### 3. SUMMARY OF THE INVENTION

The present invention relates to methods for preventing cancer by administration of fusion cells formed by fusion of dendritic cells and pre-cancerous non-dendritic cells, which fusion cells may be also be administered in combination with a molecule which stimulates a CTL and/or humoral immune response. The invention is based, in part, on the discovery and demonstration that fusion cells of dendritic cells (DCs) and non-dendritic cells having a specific allele predisposing the host organism to cancer, results in a potentiated immune response against development of that cancer. Moreover, when such fusion cells are administered in combination with a molecule which stimulates a CTL and/or humoral immune response, i.e., an immune activator, such as, for example, a cytokine, an enhanced anti-tumor response is obtained. Such fusion cells combine the vigorous immunostimulatory effect of dendritic cells with the specific antigenicity of such tumor cells, thereby eliciting a strong, specific immune response, which is further enhanced by the co-administration of an immune activator.

In one embodiment of the invention, a method for preventing or treating cancer is provided wherein fusion cells that comprise dendritic cells and pre-cancerous non-dendritic cells are administered to a patient in need of treatment. In another embodiment, fusion cells comprising dendritic cells and pre-cancerous non-dendritic cells are co-administered with a cytokine or other molecule which stimulates a CTL and/or humoral immune response, thereby significantly enhancing the effectiveness of the therapeutic treatment.

In another embodiment, the invention provides a method of preventing cancer in a mammal, which comprises administering to a mammal in need of such prevention a therapeutically effective amount of a fusion cell formed by the fusion of a dendritic cell and a pre-cancerous non-dendritic cell. In a preferred embodiment, the fusion cells are administered in combination with a molecule which stimulates a CTL and/or humoral immune response. In another aspect of this embodiment, the co-stimulator of a CTL and/or humoral immune response is also provided by transforming or transfecting the fusion cells with genetic material that encodes the co-stimulator.

In another embodiment, the invention provides a method of preventing cancer in a mammal, said method comprising administering to a mammal in need of said prevention an effective amount of fusion cells, wherein each said fusion cell is formed by the fusion of a dendritic cell and a pre-cancerous non-dendritic cell and shares at least one MHC class I allele with said mammal, and wherein said pre-cancerous non-dendritic cell displays at least one antigen having the antigenicity of an antigen specific to said cancer. In a specific embodiment of this method, the pre-cancerous non-dendritic cell is the same cell type as the cancer to be prevented. In another specific embodiment, the method further comprises administration of a molecule that stimulates a humoral immune response or a cytotoxic T cell immune response. In one embodiment, said molecule is a cytokine. In one embodiment, the cytokine is interleukin-12. In another embodiment, the dendritic cell is obtained from human blood monocytes. In another embodiment, said pre-cancerous non-dendritic cell is obtained from a primary culture of pre-cancerous cells derived from said mammal. In another embodiment, said dendritic cells are autologous to said mammal. In another embodiment, said dendritic cells and said non-dendritic cells are both autologous to said mammal, and an immunostimulatory molecule such as IL-12 is co-administered with the fusion cells. In another embodiment, said dendritic cells are allogeneic to the mammal. In another embodiment, said dendritic cells are allogeneic to the mammal and wherein said pre-cancerous non-dendritic cells have the same class I MHC haplotype as the mammal. In another embodiment, said pre-cancerous non-dendritic cells are recombinant cells transformed with a nucleic acid encoding an antigen that displays the antigenicity of a tumor-specific antigen. In another embodiment, mammal is a human. In another embodiment, the mammal is selected from the group consisting of a cow, a horse, a sheep, a pig, a fowl, a goat, a cat, a dog, a hamster, a mouse and a rat.

In another embodiment of this method, said cancer is selected from the group consisting of renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenstrom's macroglobulinemia, and heavy chain disease.

In another embodiment, the invention provides a method of treating a pre-cancerous lesion in a mammal, said method comprising administering to a mammal in need of said treatment a therapeutically effective amount of fusion cells, wherein each said fusion cell is formed by the fusion of a dendritic cell and a pre-cancerous non-dendritic cell and shares at least one MHC class I allele with said mammal, and wherein said pre-cancerous non-dendritic cell displays at least one antigen having the antigenicity of an antigen specific to said pre-cancerous lesion. In a specific embodiment, said pre-cancerous non-dendritic cell is the same cell type as said pre-cancerous lesion. In another specific embodiment, said pre-cancerous non-dendritic cell is isolated from said pre-cancerous lesion. In another specific embodiment, the method further comprises administration of a molecule that stimulates a humoral immune response or a cytotoxic T cell immune response. In one embodiment, said molecule is a cytokine. In one embodiment, the cytokine is interleukin-12. In another embodiment, the dendritic cell is obtained from human blood monocytes. In another embodiment, said pre-cancerous non-dendritic cell is obtained from a primary culture of pre-cancerous cells derived from said mammal. In another embodiment, said dendritic cells are autologous to said mammal. In another embodiment, said dendritic cells and said non-dendritic cells are both autologous to said mammal, and an immunostimulatory molecule such as IL-12 is co-administered with the fusion cells. In another embodiment, said dendritic cells are allogeneic to the mammal. In another embodiment, said dendritic cells are allogeneic to the mammal and wherein said pre-cancerous non-dendritic cells have the same class I MHC haplotype as the mammal. In another embodiment, said pre-cancerous non-dendritic cells are recombinant cells transformed with a nucleic acid encoding an antigen that displays the antigenicity of a tumor-specific antigen. In another embodiment, mammal is a human. In another embodiment, the mammal is selected from the group consisting of a cow, a horse, a sheep, a pig, a fowl, a goat, a cat, a dog, a hamster, a mouse and a rat.

In another embodiment, said pre-cancerous lesion is a precursor of a cancer selected from the group consisting of renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenström's macroglobulinemia, and heavy chain disease.

The invention further encompasses a method for fusing human dendritic cells and pre-cancerous non-dendritic human cells comprising subjecting a population of dendritic cells and a population of pre-cancerous non-dendritic cells to conditions that promote cell fusion. In one embodiment, said pre-cancerous non-dendritic cells are autologous to said dendritic cells. In another embodiment, the cell fusion is accomplished by electrofusion. In another embodiment, the method further comprising the step of inactivating the population of fusion cells. In another embodiment, the inactivating the population of fusion cells is accomplished by γ irradiating the cells.

The invention further provides a kit comprising, in one or more containers, a population of dendritic cells and instructions for fusing said dendritic cells with pre-cancerous non-dendritic cell for administration to a mammal in need thereof In one embodiment, the kit further comprises a molecule that stimulates an immune response selected from the group consisting of humor immune responses, cytotoxic T cell responses, and combinations thereof, and instructions for use of the kit for preventing or treating cancer. In one embodiment, the molecule is a cytokine. In another embodiment, the cytokine is IL-12. In another embodiment, the kit further comprises a cuvette suitable for electrofusion. In another embodiment, the dendritic cells are cryopreserved.

In another embodiment, the invention provides a pharmaceutical composition comprising a fusion cell comprising a dendritic cell fused to a pre-cancerous non-dendritic cell. In one embodiment, the pre-cancerous non-dendritic cell is freshly isolated or obtained from a primary cell culture. In another embodiment, the pharmaceutical composition further comprises a molecule that stimulates an immune response selected from the group consisting of humor immune responses, cytotoxic T cell responses, and combinations thereof. In another embodiment, the molecule is a cytokine. In another embodiment, the molecule is IL-12. In another embodiment, the dendritic cell is autologous to the pre-cancerous non-dendritic cell. In another embodiment, the dendritic cell is a human cell. In another embodiment, the pre-cancerous non-dendritic cell is a human cell. In another embodiment, the pre-cancerous non-dendritic cell is the same cell type as the cancer to be prevented. In another embodiment, the pre-cancerous non-dendritic cell is the same cell type as the pre-cancerous lesion to be treated. In another embodiment, the pre-cancerous non-dendritic cell is isolated from a pre-cancerous lesion autologous to the mammal, and wherein the pre-cancerous lesion is a precursor of a cancer to be prevented. In another embodiment, the pre-cancerous non-dendritic cell is isolated from a pre-cancerous lesion of the mammal that is to be treated with said composition.

In another embodiment, the invention provides for fusion cells comprising a dendritic cell that is fused to a pre-cancerous non-dendritic cell. In a preferred embodiment, both the dendritic and pre-cancerous non-dendritic cells are human. The present invention also encompasses a population of such fusion cells, wherein at least 10%-15% of the cells are fused, and preferably 20%-30% of the cells are fused.

As used herein, a compound, such as a cytokine, is said to be “co administered” or administered in “combination” with another compound, such as a fusion cell, when either the physiological effects of both compounds, or the elevated serum concentration of both compounds can be measured simultaneously. With compounds that increase the level of endogenous cytokine production, the serum concentration of the endogenously produced cytokine and the other administered agent (i.e., fusion cell), can also be measured simultaneously when “co-administered” or in “combination.” Thus, compounds may be administered either simultaneously, as separate or mixed compositions, or they may be administered sequentially provided that an elevation of their levels in serum can be measured simultaneously at some point during administration.

Unless otherwise stated the terms “combination therapy” and “combination treatments” are used herein to describe a therapeutic regimen involving co-administration of the subject fusion cells and a molecule which stimulates a CTL response and/or humoral immune response, which results in preventing cancer, which can be measured, for example, by demonstration of a reduction in the number of tumor cells that form, or by the failure to develop pre-cancerous lesions or tumors in a patient genetically predisposed to do so, and by the failure, or reduced rate of progression, of one or more pre-cancerous lesions to develop into tumors.

In another embodiment, the invention provides a kit comprising, in one or more containers, a sample containing a population of dendritic cells and instructions for its use in preventing cancer. In another embodiment, the kit further comprising a cuvette suitable for electrofusion. In another embodiment, the dendritic cells are cryopreserved. In a further embodiment, the kit comprises a molecule that stimulates a humoral immune response and/or a cytotoxic T cell response. In a more preferred embodiment the stimulatory molecule is a cytokine such as, but not limited to interleukin-12.

### 4. BRIEF DESCRIPTION OF THE FIGURES

FIG. 1A-C. Macroscopic View of the Upper Ileum of APC1309 Mouse. Upper ilei of 10-week-old APC-1309 mice are shown, with tumors identified as black dots on the mucosa. The upper (A), middle (B), and bottom (C) panels depict ilei from an untreated mouse, a mouse administered fusion cells, and a mouse administered fusion cells and IL-12.

FIG. 2. Number of Gastrointestinal Tumors Developed in APCl309 Mice. A control group often APC1309 mice were sacrificed at six weeks of age to provide a baseline value for the number of gastrointestinal tumors of APC-1309 mice. Additional groups of ten APC 1309 mice each were sacrificed at ten weeks of age and the number of gastrointestinal tumors were determined for untreated APC 1309 mice, APC 1309 mice administered IL-12 alone, APC1309 mice administered fusion cells, and APC1309 mice administered fusion cells and IL-12. Each column depicts the mean ± SD (error bar) of the number of tumors for each group; the symbol (*) indicates a P value of <0.0001, while the symbol (**) indicates a P-value of <0.0039. Tumors were counted under a dissection microscope.

FIG. 3. Relationship between the Number of Gastrointestinal Tumors and the Median Fluorescence of Tumor Cells Incubated with Serum. Gastrointestinal tumors were counted as in FIG. 1 for untreated APC 1309 mice, APC 1309 mice administered fusion cells, and APC1309 mice administered fusion cells and IL-12. Tumor cells (2×10⁵) were incubated with serum, washed and incubated with FITC-conjugated rat-anti-mouse immunoglobulin antibody. Fluorescent intensity of the labeled cells was determined by fluorescence-activated cell sorting (FACS) analysis. Each symbol represents the number of gastrointestinal tumors and the median fluorescent intensity shown by the individual mouse. ▪: APC1309 mice untreated; ◯: APC1309 mice treated with fusion cells; [symbol] : APC1309 mice administered fusion cells and IL-12.

FIG. 4A-B. (A) Effect of Serum Dilution on Median Fluorescent Intensity. The median fluorescent intensities shown by tumor cells incubated with sera and then FITC-conjugated rat anti-mouse immunoglobulin antibody were determined as described in the legend to FIG. 3 except that diluted sera were used. ▪, &Rectversolid;: sera from untreated mice; x, [symbol] : sera from mice administered fusion cells and IL-12. (B) Effect of Incubation of Serum with Tumor Cells on Median Fluorescent Intensity. Serum of an APC1309 mouse that had been treated with fusion cells and IL-12 was diluted one hundred-fold with PBS, incubated with tumor cells (1×10⁶), and then centrifuged, thereby removing cell-bound antibodies from the serum. Fluorescent intensity of serum and the supernatant was then determined as in FIG. 3. The top and middle panels provide the fluorescent intensity histogram exhibited by the serum and the supernatant, respectively. The bottom panel depicts the fluorescent intensity histogram exhibited by tumor cells incubated with 100-fold diluted serum from an untreated mouse.

FIG. 5. Effect of Serum from APC1309 Mice Administered Fusion Cells and IL-12 on Tumor Cell Growth In Vitro. Tumor cells (1×10⁵) were cultured for 48 hours at 37° C. in the absence [symbol] or the presence of serum from an untreated APC1309 mouse [symbol] serum from an APC1309 mouse administered fusion cells and IL-12 [symbol] , heat-inactivated serum from an untreated APC 1309 mouse [symbol] and heat-inactivated serum from an APC1309 mouse administered fusion cells and IL-12 [symbol] . Sera were added to give a final dilution of 300. Each column represents the mean ± SD (error bars) of the total number of viable cells in three wells.

FIG. 6A-C. Immunohistochemical Analysis of Lymphocytes Infiltrating into Gastrointestinal Tumors of APC1309 Mice Administered Fusion Cells and IL-12. Frozen sections of intestinal tumors were stained for CD4, CD45R, and immunoglobulin with FITC-conjugated rat anti-mouse CD45R antibody (PharMingen, San Diego) and FITC-conjugated rat anti-mouse immunoglobulin antibody (PharMingen, San Diego): (A) untreated APC1309 mice, and (B) APC1309 mice administered fusion cells and IL-12. Note that abundant immunoglobulin positive cells, CD45R⁺ cells and some CD4⁺ cells infiltrated into the tumor tissue of APC1309 mice administered fusion cells and IL-12. (C) Infiltration of CD45R⁺ and Immunoglobulin-positive Cells into the Intestinal Lymphoid Follicle of an Apc1309 Mouse Administered Fusion Cells and IL-12. Frozen sections of the intestinal lymphoid follicle of an APC1309 mouse administered fusion cells and IL-12, were stained for CD45R and immunoglobulin with PE-conjugated rat anti-mouse immunoglobulin antibody (PharMingen, San Diego, Calif.). Note that abundant CD45R⁺ and immunoglobulin-positive cells infiltrated into the intestinal lymphoid follicle.

### 5. DETAILED DESCRIPTION OF THE INVENTION

The invention provides methods for the prevention of cancer, in which fusion cells formed by fusing dendritic cells with pre-cancerous non-dendritic cells. A prophylactic amount of such fused cells is administered to a subject in need of such prevention. In certain embodiments, such fused cells are administered in combination with a therapeutically effective amount of a molecule which stimulates a humoral immune response and/or a cytotoxic T-lymphocyte response (CTL). In a preferred embodiment, the invention relates to methods comprising administration of a therapeutically effective amount of fusion cells in combination with a cytokine such as, but not limited to, IL-12.

According to the methods described herein, dendritic cells are fused to pre-cancerous non-dendritic cells containing an antigen characteristic of the cancer to be prevented. The resulting fusion cells comprising dendritic cells and pre-cancerous non-dendritic cells are used as a potent composition for the prevention of tumors comprising that antigen that develop from pre-cancerous non-dendritic cells comprising that antigen.

In one embodiment, this approach is advantageous when a specific antigen is not readily identifiable, as is generally the case with respect to pre-cancerous cells. For prevention of human cancer, for example, pre-cancerous non-dendritic cells are obtained directly from a pre-cancerous lesion of a patient, e.g. by biopsy. In this instance, fusion cells formed from such pre-cancerous non-dendritic cells, and compositions comprising such fusion cells, are highly specific forthe cancer to be prevented.

Described below, are methods for the prevention of cancer. In particular, Sections 5.1, 5.2, and 5.3 describe the pre-cancerous non-dendritic cells, dendritic cells, and fusion cells formed by fusion of pre-cancerous non-dendritic cells with dendritic cells, respectively, that are used in the invention, as well as methods for the isolation, preparation, and/or generation of those cells. Target cancers that can be treated or prevented using such compositions are described below in Sections 5.4 and 5.5.

5.1 Pre-Cancerous Non-Dendritic Cells

A pre-cancerous non-dendritic cell of the present invention can be any non-dendritic cells bearing at least one allele that distinguishes the pre-cancerous cell from a normal cell. Such non-dendritic cells may be isolated from a variety of sources, such as, but not limited to, a pre-cancerous lesion of the patient in need of preventive treatment. The pre-cancerous non-dendritic cells may also be from a primary cell culture that may be autologous, syngeneic, or allogeneic to the patient, depending on the source of the dendritic cells to be used in preparation of the fusion cells. Methods for isolation and preparation of such non-cancerous non-dendritic cells are described in detail hereinbelow.

The source of the precancerous non-dendritic cells is selected according to the cancer to be prevented. Preferably, the non-dendritic cells are autologous to the patient being treated. Since whole non-dendritic cells are used in the present methods, it is not necessary to isolate, characterize, or even know the identities of, these antigens prior to performing the present methods. However, any non-dendritic cell can be used as long as the cell comprises at least one antigen that is specific to the target cells. In one embodiment, where the dendritic cell is allogeneic to the patient, the non-dendritic cell may have, in addition, at least one MHC I allele that is of the same class I MHC haplotype as the mammal being treated. In another embodiment, where the dendritic cell is autologous to the patient, the non-dendritic cell may be an allogeneic or autologous to the mammal being treated.

For prevention of cancer, the non-dendritic cell is a pre-cancerous non-dendritic cell. In this embodiment, the invention provides fusion cells that express at least one antigen expressed by a pre-cancerous cell as well as a cancer cell that develops therefrom, e.g., a tumor-specific antigen or a tumor associated antigen, that is capable of eliciting an immune response against such pre-cancerous or cancer cells which develop therefrom. In one embodiment of the invention, cells isolated from pre-cancerous lesions, or pre-cancerous tissues are used for the preparation of the non-dendritic cells. Non-limiting examples of cancers that are amenable to the methods of the invention are listed in Section 5.3 and 5.6, infra.

Pre-cancerous non-dendritic cells may be isolated by surgical excision or biopsy of any precancerous legion, many of which are known in the art. In one embodiment, for example, pre-cancerous non-dendritic cells are isolated, by surgical excision or biopsy of a medically-recognized pre-cancerous lesion designated Barrett's metaplasia, which is a precursor of esophageal adenocarcinoma. This lesion is a heterologous lesion generally found in the region of the gastro-esophageal junction. Pre-cancerous cell clones isolated from such lesions exhibit genetic and biological heterogeneity including, for example, p53 mutations, pl6 mutations, and aneuploidy. These alterations are accompanied by discrete changes in cellular proliferation, differentiation, and apoptosis, which underlie a evolution from normal cell through metaplasia—dysplasia—adenocarcinoma stages by which a pre-cancerous cell develops into a tumor cell. Similarly, intestinal metaplasia of the gastric cardia have been proposed as pre-cancerous lesions of adenocarcinoma of the gastric cardia (see, e.g., Jankowski et al., 1999, Am. J. Pathol. 154(4): 965-73; Jankowski et al., 2000, Barrett's Metaplasia, Lancet 356(9247): 2079-85; Haringsma et al., 2001, Gastrointest Endosc 53(6): 642-50; and Ruol et al., 2000, Cancer 88(11): 2520-28).

In another embodiment, pre-cancerous non-dendritic cells are isolated by surgical excision or biopsy of gastrointestinal polyps which in many instances represent pre-cancerous lesions that progress, with time, to an adenocarcinoma. Methods for identification and excision of such polyps are well known in the art. Such polyps arise during the development of sporadic colorectal cancer as well as in the development and progression of the heritable diseases familial adenomatous polyposis (FAP), hereditary non-polyposis colorectal cancer (HNPCC), and juvenile polyposis (JPS) (see e.g. Souza, A, 2001, Ailment Pharmacol. Ther. 15(4): 451-62). FAP and HNPCC represent two well-defined forms of hereditary colorectal cancer: (a) familial adenomatous polyposis (FAP), which is caused by germ line mutations of adenomatous polyposis coli (APC) gene; and (b) hereditary nonpolyposis colorectal cancer (HNPCC), which is caused by germ line mutations of a mismatch repair gene (Boland C. R., Malignant tumors of the colon. In Textbook of Gastroenterology 2^{nd} Ed. (Eds. Yamada T) 1967-2026 (J. B. Lippincot Company, Philadelphia, (1995); Kinzler, et al., 1991, Science 253:661-665; Lynch et al., 1996, Cancer 78:1149-1167; Peltomaki et al., 1997, Gastroenterology 113:1146-1158). These hereditary colorectal cancers are characterized by their early onset and high mortality rate. In all FAP patients, adenomatous polyps develop at a median patient age of 16 years, and virtually all affected individuals develop cancer by a median age of 39 years (Boland C. R., Malignant tumors of the colon, in Textbook of Gastroenterology 2^{nd} Ed. (Eds. Yamada T) 1967-2026 (J. B. Lippincot Company, Philadelphia, (1995)). Mutation of APC gene is also observed in 70-80% of sporadic colon cancer patients (Nakamura Y., 1997, Nature Medicine News & Views. 3:499-500).

In still another embodiment, pre-cancerous non-dendritic cells are isolated by surgical excision or biopsy of intratubular epithelial dysplasia, which is the most common medically-recognized precursor of renal cell carcinoma. In another aspect of this embodiment, pre-cancerous non-dendritic cells are isolated, by surgical excision or biopsy of one or more of the well-documented pre-cancerous lesions of the vonHippel-Lindau syndrome. In this disease, there is an evolution from a pre-cancerous, simple cyst, through an atypical cyst with epithelial hyperplasia, and culminating in a cystic or solid renal cell carcinoma. Moreover, a developmental sequence progressing from pre-cancerous adenomatous lesions to carcinomas has also been observed in papillary renal cell carcinoma. Accordingly, such pre-cancerous adenomatous lesions are also useful sources for isolation of pre-cancerous non-dendritic cells (see e.g. VanPoppel et al., 2000, Scand. J. Urol. Nephrol. Suppl. 205: 136-65).

In another embodiment, pre-cancerous non-dendritic cells are isolated, by surgical excision or, preferably by biopsy of dysplasia detected during screening endoscopic retrograde cholangiopancreatography (ERCP) procedures. ERCP screening is indicted in instances of familial pancreatic cancer, and in instances of hereditary pancreatitis, which is associated with a 40% lifetime risk of developing pancreatic ductal adenocarcinoma (see e.g. Howes et al., 2000, Med. Clin. North Am. 84(3): 719-38; and Brentnall, 2000, Med. Clin. North Am. 84(3): 707-18).

In still another embodiment, pre-cancerous non-dendritic cells are isolated by surgical excision or by biopsy of actinic keratoses, benign nevi, and dysplasic nevi. Actinic keratoses and pre-cancerous lesions characteristic of Bowen's disease (squamous cell carcinoma in situ) provide non-cancerous cells that are precursors to the development of squamous cell carcinoma (SSC), while benign nevi, and dysplasic nevi are potential precursors of malignant melanoma (see e.g. Gloster et al., 1996, Dermatol. Surg. 22(3): 217-26; and Sober et al., 1995, Cancer 75(2 Suppl.): 645-50.

In another embodiment, pre-cancerous non-dendritic cells are isolated by surgical excision or biopsy of pre-cancerous lesions leading to breast cancer. It has been reported that atypical cystic duct (ACD) is the precancerous lesion of breast cancer based upon an observed histologic continuum between ACD and malignancy and because of the expression of the p53 protein in ACD (Kusama et. al., 2000, Pathol. Int. 50(10): 793-800). Similarly, noncomedo ductal carcinoma in situ (DCIS) lesions and especially comedo ductal carcinoma in situ lesions are associated with an elevated risk (more than eight-fold) of developing invasive breast cancer, and, therefore are sources for isolation of pre-cancerous non-dendritic cells useful in the present invention (see, e.g., Lawrence et al., 1998, Cancer Epidemiol. Biomarkers Prev. 7(1): 29-35).

In a further embodiment, pre-cancerous non-dendritic cells are isolated, by surgical excision or biopsy of high-grade prostatic intraepithelial neoplasia lesions, which are recognized pre-cancerous lesions important in neoplastic development, especially when accompanied by adjacent atypical glands (Sakr et al., 2001, Urology 57(4): 115-20; Zlotta et al., 1999, Eur. Urol. 35(5-6): 498-503; Alsikafi et al., 2001, Urology 57(2): 296-300; and Molinie, 2001, Ann. Pathol. 21(3): 245-254).

In a still further embodiment, pre-cancerous non-dendritic cells are isolated by surgical excision or biopsy of any one of at least three different lesions that are regarded as comprising pre-cancerous cells of lung cancer: (1) squamous dysplasia and carcinoma in situ; (2) atypical adenomatous hyperplasia; and (3) diffuse idiopathic pulmonary neuroendocrine cell hyperplasia (Kerr, 2001, J. Clin. Pathol. 54(4): 257-71.

In another embodiment, pre-cancerous non-dendritic cells are isolated by surgical excision or biopsy of oral leukoplakia, which can appear as a white patch on oral mucosa, that are recognized as pre-cancerous lesions which have a high probability of developing into oral cancer (Mao, 1997, Mol. Med. Today, 3(10): 442-48).

Although specific sources of pre-cancerous non-dendritic cells have been disclosed above with respect to colorectal, prostatic, esophageal, renal, pancreatic, skin, breast, lung, and oral cancers, the present invention is not to be limited to these specific embodiments. That is, as is apparent to one of ordinary skill, pre-cancerous tissues are readily characterized as hyperplasic, metaplasic, and dysplasic, and which comprise pre-cancerous cells having at least one genetic allele that distinguishes those pre-cancerous cells from normal cells. In addition, genetic tests,.which are now available and will be developed as analysis of the human genome continues, that permit rapid and precise identification of the presence of specific alleles associated with an increased risk of cancer development. Accordingly, identification and analysis of pre-cancerous tissues suitable for use as sources of pre-cancerous non-dendritic cells of the present invention are readily performed by, inter alia, oncologists and, more particularly, molecular oncologists of ordinary skill.

In certain embodiments, the pre-cancerous non-dendritic cells are not freshly isolated, but are instead cultured to select for pre-cancerous non-dendritic cells to be fused with dendritic cells and thereby prevent or limit contamination of a population of pre-cancerous cells with healthy, non-precancerous cells.

In a preferred embodiment, the pre-cancerous non-dendritic cells of the invention are isolated from a pre-cancerous lesion that is surgically removed from the mammal that will be the recipient of the fusion-cell containing compositions. Prior to use, solid pre-cancerous tissue or aggregated pre-cancerous cells should be dispersed, preferably mechanically, into a single cell suspension by standard techniques. Enzymes, such as but not limited to, collagenase and DNase may also be used to disperse cancer cells. In yet another preferred embodiment, the pre-cancerous non-dendritic cells of the invention are obtained from primary cell cultures, i.e., cultures of original cells obtained from the body.

The amount of pre-cancerous non-dendritic cells collected should be sufficient to fuse with dendritic cells to prepare enough fusion cells for the vaccines of the invention. In a preferred embodiment, 5×10⁷ pre-cancerous non-dendritic cells is used as starting material for the formation of fusion cells. In one embodiment, approximately 1×10⁶ to 1×10⁹ pre-cancerous non-dendritic cells are used for formation of fusion cells. In another embodiment, 5×10⁷ to 2×10₈ pre-cancerous non-dendritic cells are used. In yet another embodiment, 1×10⁷ to 1×10¹⁰ pre-cancerous non-dendritic cells are used. The use of other quantities of pre-cancerous non-dendritic cells for preparation of fusion cells are within the scope of the invention.

In another embodiment, suitable pre-cancerous non-dendritic cells are preferably of the same cell type as the cancer desired to be inhibited and are isolated from the recipient or, less preferably, a family member or an individual who shares at least one MHC allele, and preferably the class I MHC haplotype, with the intended recipient and who carries the pre-cancerous lesions of the cancer to be prevented.

Alternatively, if a gene encoding a tumor-specific antigen, or a tumor-associated antigen, is available, normal cells of the appropriate cell type from the intended recipient can be transformed or transfected with that gene, where that gene is heterologously expressed and provides an immunologically-detectable amount of the tumor-specific or tumor-associated antigen. Such recombinant cells can be used as a source of pre-cancerous non-dendritic cells. Optionally, more than one such antigen may be expressed in the recipient's cell in this fashion, as will be appreciated by those skilled in the art, any techniques known, such as those described in Ausubel et aL (Ausubel et al. eds., 1989, Current Protocols in Molecular Biology, Greene Publishing Associates and Wiley Interscience, New York), may be used to perform the transformation or transfection and subsequent recombinant expression of the gene(s) encoding the tumor-specific or tumor-associated antigen(s) in recipient's cells. These non-dendritic cells bearing one or more MHC molecules in common with the recipient are suitable for use in methods for formation of fusion cells according to the present invention.

Where allogeneic dendritic cells are to be used, pre-cancerous non-dendritic cells used for generation of fusion cells with allogeneic dendritic cells must have at least one common MHC allele in order to elicit an immune response in the mammal. Most preferred are pre-cancerous non-dendritic cells derived from the intended recipient, i.e., the pre-cancerous non-dendritic cells are autologous to the patient to whom the fusion cells of the present invention are to be administered. In one embodiment, non-dendritic cells that are nonautologous, but share at least one MHC allele with the target pre-cancerous cells or cancer cells of the recipient may be used. If the pre-cancerous non-dendritic cells are obtained from the same or from a syngeneic individual, such cells will have the same class I MHC haplotype. If they are not all obtained from the same subject or a syngeneic source, the MHC haplotype can be determined by standard HLA typing techniques well known in the art, such as serological tests and DNA analysis of the MHC loci. An MHC haplotype determination does not need to be undertaken prior to carrying out the procedure for generation of the fusion cells of the invention.

Non-dendritic pre-cancerous cells, such as cells containing an antigen having the antigenicity of a cancer cell can be identified and isolated by any method known in the art. For example, pre-cancerous non-dendritic cells can be identified by morphology, enzyme assays, proliferation assays, or the presence of cancer-causing viruses. If the characteristics of the antigen of interest are known, pre-cancerous non-dendritic cells can also be identified or isolated by any biochemical or immunological methods known in the art. For example, pre-cancerous non-dendritic cells can be isolated by surgery, endoscopy, other biopsy techniques, affinity chromatography, and fluorescence activated cell sorting (e.g., with fluorescently tagged antibody against an antigen expressed by the pre-cancerous non-dendritic cells).

There is no requirement that a clonal or homogeneous or purified population of pre-cancerous non-dendritic cells be used. A mixture of cells can be used, provided that a substantial number of cells in the mixture contain the antigen or antigens of the pre-cancerous cells being targeted. In a specific embodiment, the pre-cancerous non-dendritic cells and/or dendritic cells are purified.

5.2 Dendritic Cells

Dendritic cells (DC) can be isolated or generated from blood or bone marrow, or secondary lymphoid organs of the subject, such as but not limited to spleen, lymph nodes, tonsils, Peyer's patch of the intestine or bone marrow, by any of the methods known in the art. In a preferred embodiment, the dendritic cells used in the methods of the invention are terminally differentiated dendritic cells. In one embodiment, dendritic cells are isolated from human blood monocytes. In certain embodiments, the dendritic cells are autologous to the subject to whom the fusion cells of the present invention are to be administered. In alternative embodiments, the dendritic cells are allogeneic to the subject to whom the fusion cells of the present invention are to be administered.

Immune cells obtained from such sources typically comprise predominantly recirculating lymphocytes and macrophages at various stages of differentiation and maturation. Dendritic cell preparations can be enriched by standard techniques (see e.g., Current Protocols in Immunology, 7.32.1-7.32.16, John Wiley and Sons, Inc., 1997). In one embodiment, for example, dendritic cells may be enriched by depletion of T cells and adherent cells, followed by density gradient centrifugation. Dendritic cells may optionally be further purified by sorting of fluorescently-labeled cells, or by using anti-CD83 mAb magnetic beads.

Alternatively, a high yield of a relatively homogenous population of dendritic cells can be obtained by treating dendritic cell progenitors present in blood samples or bone marrow with cytokines, such as granulocyte-macrophage colony stimulating factor (GM-CSF) and interleukin 4 (IL-4). Under such conditions, monocytes differentiate into dendritic cells without cell proliferation. Further treatment with an agent such as, but not limited to, TNFα stimulates terminal differentiation of dendritic cells.

In certain embodiments, the yield of dendritic cells can be increased by administering an effective amount of FLT3 ligand and to the individual from whom the dendritic cells are to be isolated (see, e.g., Fong et al., 2000, Proc. Natl. Sci. USA 98(15):8809-14).

By way of example but not limitation, dendritic cells are obtained from blood monocytes according to standard methods (see, e.g., Sallusto et al., 1994, J. Exp. Med. 179:1109-1118). Leukocytes from healthy blood donors are collected by leukapheresis pack or buffy coat preparation using Ficoll-Paque density gradient centrifugation and plastic adherence. If mature dendritic cells are desired, the following protocol may be used to culture dendritic cells. Cells are allowed to adhere to plastic dishes for 4 hours at 37° C. Nonadherent cells are removed and adherent monocytes are cultured for 7 days in culture media containing 0.1 μg/ml granulocyte-macrophage colony stimulating factor and 0.05μg/ml interleukin-4. In order to prepare dendritic cells, tumor necrosis factor-α is added on day 5 and cells are collected on day 7.

Dendritic cells obtained in this way characteristically express the cell surface marker CD83. In addition, such cells characteristically express high levels of MHC class II molecules, as well as cell surface markers CD1α, CD40 , CD86, CD54, and CD80, but lose expression of CD14. Other cell surface markers characteristically include the T cell markers CD2 and CD5, the B cell marker CD7 and the myeloid cell markers CD13, CD32 (FcγR II), CD33, CD36, and CD63, as well as a large number of leukocyte-associated antigens.

Optionally, standard techniques such as morphological observation and immunochemical staining, can be used to verify the presence of dendritic cells. For example, the purity of dendritic cells can be assessed by flow cytometry using fluorochrome-labeled antibodies directed against one or more of the characteristic cell surface markers noted above, e.g., CD83, HLA-ABC, HLA-DR, CD1α, CD40, and/or CD54. This technique can also be used to distinguish between and immature dendritic cells, using fluorochrome-labeled antibodies directed against CD14, which is present in immature, but not in mature, differentiated dendritic cells.

5.3 Generation of Fusion Cells

Pre-cancerous non-dendritic cells can be fused to dendritic cells as follows. Cells are sterile-washed and fused according to any cell fusion technique in the art, provided that the fusion technique results in a mixture of fused cells suitable for injection into a mammal for prevention of cancer. Preferably, electrofusion is used. Electrofusion techniques are well known in the art (Stuhler and Walden, 1994, Cancer Immunol. Immunother. 39: 342-345; see Chang et al. (eds.), Guide to Electroporation and Electrofusion. Academic Press, San Diego, 1992).

In a preferred embodiment, the following protocol is used. In the first step, approximately 5×10⁷ pre-cancerous non-dendritic cells and 5×10⁷ dendritic cells are suspended in 0.3 M glucose and transferred into an electrofusion cuvette. The sample is dielectrophoretically aligned to form cell-cell conjugates by pulsing the cell sample at 100 V/cm for 5-10 sec. Optionally, alignment may be optimized by applying a drop of dielectrical wax onto one aspect of the electroporation cuvette to ‘inhomogenize’ the electric field, thus directing the cells to the area of the highest field strength. In a second step, a fusion pulse is applied. Various parameters may be used for the electrofusion. For example, in one embodiment, the fusion pulse may be from a single to a triple pulse. In another embodiment, electrofusion is accomplished using from 500 to 1500V/cm, preferably, 1,200V/cm at about 25 μF.

In a preferred embodiment, the pre-cancerous non-dendritic cells are autologous to the patient to whom the fusion cells of the present invention are to be administered. In another preferred embodiment, the dendritic cells are autologous to the patient to whom the fusion cells of the present invention are to be administered. In an even more preferred embodiment, both the pre-cancerous non-dendritic cells and the dendritic cells are autologous to the patient to whom the fusion cells of the present invention are administered.

In another embodiment, the following protocol is used. First, bone marrow is isolated and red cells lysed with ammonium chloride (Sigma, St. Louis, Mo.). Lymphocytes, granulocytes and dendritic cells are depleted from the bone marrow cells and the remaining cells are plated in 24-well culture plates (1×10⁶ cells/well) in RPMI 1640 medium supplemented with 5% heat-inactivated FBS, 50 μM 2-mercaptoethanol, 2 mM glutamate, 100 U/ml penicillin, 100 pg/ml streptomycin, 10 ng/ml recombinant granulocyte-macrophage colony stimulating factor (GM-CSF; Becton Dickinson, San Jose, Calif.) and 30 U/ml recombinant interleukin-4 (IL-4; Becton Dickinson). Second, on day 5 of culture, nonadherent and loosely adherent cells are collected and replated on 100-mm petri dishes (1×10⁶ cells/mi; 10 ml/dish). Next, GM-CSF and IL-4 in RPMI medium are added to the cells and 1×10⁶ DCs are mixed with 3×10⁶ irradiated (50 Gy, Hitachi MBR-1520R, dose rate: 1.1 Gy/min) pre-cancerous non-dendritic cells. After 48 hr, fusion is initiated by adding dropwise over 60 sec, 500μl of a 50% solution of polyethylene glycol (PEG 1500; Sigma, St. Louis, Mo.). The fusion is stopped by stepwise addition of 30 ml. of serum-free RPMI medium. Fusion cells are plated in 100-mm petri dishes in the presence of GM-CSF and IL-4 in RPMI medium for 48 hours.

In another embodiment, the dendritic cell and the pre-cancerous non-dendritic cell are fused as described above. Subsequently, the fused cells are transformed or transfected with genetic material which encodes a molecule which stimulates a CTL and/or humoral immune response. In a preferred embodiment, the genetic material is mRNA encoding IL-12. Preferred methods of transfection include electroporation or transformation or transfection in the presence of cationic polymers.

The extent of fusion cell formation within a population of pre-cancerous non-dendritic cells and dendritic cells can be determined by a number of diagnostic techniques known in the art. In one embodiment, for example, hybrids are characterized by labeling dendritic cells and pre-cancerous non-dendritic cells with red and green intracellular fluorescent dyes, respectively, and detection the emission of both colors. Samples of dendritic cells without pre-cancerous non-dendritic cells, and tumor cells without dendritic cells can be used as negative controls, as well as a mixture of non-fused pre-cancerous non-dendritic cells and dendritic cells.

Before administration of fusions cells (with or without the co-administration of an immunostimulatory molecule) to a mammal, the fusion cells are inactivated, for example, by irradiation, to prevent proliferation of the fusion cells or the pre-cancerous non-dendritic cells. Preferably, the fusion cell population is irradiated at 200 Gy, and injected without further selection. In one embodiment, the fusion cells prepared by this method comprise approximately 10 and 20% of the total cell population. In yet another embodiment, the fusion cells prepared by this method comprise approximately 5 to 50% of the total cell population.

5.4 Immune Cell Activating Molecules

The present invention provides a composition which comprises first, a fusion cell derived from the fusion of a dendritic and pre-cancerous non-dendritic cell, and in certain embodiments, further comprise a cytokine or other molecule which can stimulate or induce a cytotoxic T cell (CTL) response and/or a humoral response.

In a preferred embodiment, the CTL stimulating molecule is IL-12. IL-12 plays a major role in regulating the migration and proper selection of effector cells in an immune response. The IL-12 gene product generally polarizes the immune response toward the TH₁ subset of T helper cells and strongly stimulates CTL activity. As elevated doses of IL-12 exhibits toxicity when administered systemically, IL-12 is preferably administered locally. Additional modes of administration are described below in Section 5.7.1.

Expression of IL-12 receptor β2 (IL-12R-β2) is necessary for maintaining IL-12 responsiveness and controlling TH₁ lineage commitment. Furthermore, IL-12 signaling results in STAT4 activation, i.e., measured by an increase of phosphorylation of STAT4, and interferon-γ (IFN-γ) production. Thus, in one embodiment, the present invention contemplates the use of a molecule, which is not IL-12, which can activate STAT4, for example a small molecule activator of STAT4 identified by the use of combinatorial chemistry.

In an alternative embodiment, the immune stimulating molecule is IL-18. In yet another embodiment, the immune stimulating molecule is IL-15. In yet another embodiment, the immune stimulating molecule is interferon-γ.

In another embodiment, the patient to be treated is administered any combination of molecules or cytokines described herein which stimulate or induce a CTL and/or a humoral immune response.

In a less preferred embodiment, to increase the cytotoxic T-cell pool, i.e., the TH₁ cell subpopulation, anti-IL-4 antibodies can be added to inhibit the polarization of T-helper cells into TH₂ cells, thereby creating selective pressure toward the TH₁ subset of T-helper cells. Further, anti-IL-4 antibodies can be administered concurrent with the administration of IL-12, to induce the TH cells to differentiate into TH₁ cells. After differentiation, cells can be washed, resuspended in, for example, buffered saline, and reintroduced into a patient via, preferably, intravenous administration.

In another embodiment, to enhance a humoral response, IL-4 is added to stimulate production of TH₂ helper T-cells and promote synthesis of antibodies that specifically bind to the pre-cancerous cells of the treated individual.

The present invention also pertains to variants of the above-described interleukins. Such variants have an altered amino acid sequence which can function as agonists (mimetics) to promote a CTL and/or humoral immune response. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a molecule capable of stimulating a CTL and/or humoral immune response can be identified by screening combinatorial libraries of mutants, e.g., truncation mutants, for agonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of IL-12 from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem., 53:323; Itakura et al., 1984, Science, 198:1056; Ike et al., 1983, Nucleic Acid Res., 11:477).

In addition, libraries of fragments of the coding sequence of an interleukin capable of promoting a CTL and/or humoral immune response can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of an interleukin capable of promoting a CTL and/or humoral immune response (Arkin and Yourvan, 1992, Proc. Natl. Acad. Sci. USA, 89:7811-7815; Delgrave et al., 1993, Protein Engineering, 6(3):327-331).

5.5 Assays for Measuring an Immune Response

The fusion cell-cytokine compositions can be assayed for immunogenicity using any method known in the art. By way of example but not limitation, one of the following procedures can be used.

A humoral immune response can be measured using standard detection assays including but not limited to an ELISA, to determine the relative amount of antibodies which recognize the target antigen in the sera of a treated subject, relative to the amount of antibodies in untreated subjects. A CTL response can be measured using standard immunoassays including chromium release assays as described herein. More particularly, a CTL response is determined by the measurable difference in CTL activity upon administration of a stimulator, relative to CTL activity in the absence of a stimulator.

5.5.1 MLTC Assay

The fusion cell and fusion cell, cytokine-containing compositions may be tested for immunogenicity using a mixed lymphocyte T cell culture (MLTC) assay. For example, 1×10⁷ fusion cells are γ-irradiated, and mixed with T lymphocytes. At various intervals the T lymphocytes are tested for cytotoxicity in a 4 hour ⁵¹Cr-release assay (see Palladino et al., 1987, Cancer Res. 47:5074-5079). In this assay, the mixed lymphocyte culture is added to a target cell suspension to give different effector:target (E:T) ratios (usually 1:1 to 40:1). The target cells are prelabelled by incubating 1×10⁶ target cells in culture medium containing 500 μCi ⁵¹Cr/ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelletted by centrifugation at 200×g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5%.

5.5.2 Antibody Response Assay

In one embodiment of the invention, the immunogenicity of fusion cells is determined by measuring antibodies produced in response to the vaccination, by an antibody response assay, such as an enzyme-linked immunosorbent assay (ELISA) assay. Methods for such assays are well known in the art (see, e.g, Section 2.1 of Current Protocols in Immunology, Coligan et al (eds.), John Wiley and Sons, Inc. 1997). In one mode of the embodiment, microtitre plates (96-well Immuno Plate II, Nunc) are coated with 50 μl/well of a 0.75 μg/ml solution of a purified pre-cancerous cell used in the composition in PBS at 4° C. for 16 hours and at 20° C. for 1 hour. The wells are emptied and blocked with 200 μl PBS-T-BSA (PBS containing 0.05% (v/v) TWEEN 20 and 1% (w/v) bovine serum albumin) per well at 20° C. for 1 hour, then washed 3 times with PBS-T. Fifty μl/well of plasma or CSF from a vaccinated animal (such as a model mouse or a human patient) is applied at 20° C. for 1 hour, and the plates are washed 3 times with PBS-T. The antigen antibody activity is then measured calorimetrically after incubating at 20° C. for 1 hour with 50 μl/well of sheep anti-mouse or anti-human immunoglobulin, as appropriate, conjugated with horseradish peroxidase diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a photometer at 492 nm (ref. 620 nm), using standard techniques.

5.5.3 Cytokine Detection Assays

The CD4⁺T cell proliferative response to the fusion cell-cytokine composition may be measured by detection and quantitation of the levels of specific cytokines. In one embodiment, for example, intracellular cytokines may be measured using an IFN-γ detection assay to test for immunogenicity of the fusion cell-cytokine composition. In an example of this method, peripheral blood mononuclear cells from a patient treated with the fusion cell-cytokine composition are stimulated with peptide antigens such as mucin peptide antigens or Her2/neu derived epitopes. Cells are then stained with T cell-specific labeled antibodies detectable by flow cytometry, for example FITC-conjugated anti-CD8 and PerCP-labeled anti-CD4 antibodies. After washing, cells are fixed, permeabilized, and reacted with dye-labeled antibodies reactive with human IFN-γ (PE-anti-IFN-γ). Samples are analyzed by flow cytometry using standard techniques.

Alternatively, a filter immunoassay, the enzyme-linked immunospot assay (ELISPOT) assay, may be used to detect specific cytokines surrounding a T cell. In one embodiment, for example, a nitrocellulose-backed microtiter plate is coated with a purified cytokine-specific primary antibody, i.e., anti-IFN-γ, and the plate is blocked to avoid background due to nonspecific binding of other proteins. A sample of mononuclear blood cells, containing cytokine-secreting cells, obtained from a patient vaccinated with fusion cells or fusion cells and an immune stimulator such as a cytokine composition, is diluted into the wells of the microtitre plate. A labeled, e.g., biotin-labeled, secondary anti-cytokine antibody is added. The antibody-cytokine complex can then be detected, e.g. by enzyme-conjugated streptavidin, and cytokine-secreting cells will appear as “spots” by visual, microscopic, or electronic detection methods.

5.5.4 Tetramer Staining Assay

In another embodiment, the “tetramer staining” assay (Altman et al., 1996, Science 274: 94-96) may be used to identify antigen-specific T-cells. In one embodiment, an MHC molecule containing a specific peptide antigen, such as a tumor-specific antigen, is multimerized to make soluble peptide tetramers and labeled, for example, by complexing to streptavidin. The MHC complex is then mixed with a population of T cells obtained from a patient treated with a fusion cell composition. Biotin is then used to stain T cells which express the antigen of interest, i.e., the tumor-specific antigen.

Cytotoxic T-cells are immune cells which are CD8 positive and have been activated by antigen presenting cells (APCs), that have processed and are displaying an antigen of a target cell. The antigen presentation, in conjunction with activation of co-stimulatory molecules such as B-7/CTLA-4 and CD40, leads to priming of the T-cell against the target, resulting in destruction of cells expressing the antigen.

Cytotoxic T-cells, generally characterized as expressing CD8, also secreted TNF-β, perforin, and IL-2. A cytotoxic T cell response can be measured in various assays, including but not limited to increased target cell lysis in ⁵¹ Cr release assays using T-cells from treated subjects, in comparison to T-cells from untreated subjects, as shown in the examples herein, as well as measuring an increase in the levels of IFN-γ and IL-2 in treated subjects relative to untreated subjects.

5.6 Target Cancers

The cancers and oncogenic diseases that can be prevented, as well as the pre-cancerous lesions, which lead to the development of those cancers and oncogenic diseases, that can be prevented and treated, using the fusion cells of the present invention include, but are not limited to: human sarcomas and carcinomas, e.g., renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

5.7 Pharmaceuticals Preparations and Methods of Administration

The composition formulations of the invention comprise an effective immunizing amount of the fusion cells which are to be administered either without or with one or more molecules, such as but not limited to cytokines, that are capable of stimulating a CTL and/or humoral immune response.

Suitable preparations of fusion cell or fusion cell-cytokine compositions include injectable formulations that are, preferably, liquid solutions.

Many methods may be used to introduce the composition formulations of the invention; these include but are not limited to subcutaneous injection, intralymphatically, intradermal, intramuscular, intravenous, and via scarification (scratching through the top layers of skin, e.g., using a bifurcated needle). Preferably, fusion cell and fusion cell-cytokine compositions are injected intradermally.

In addition, if desired, the composition preparation may also include minor amounts of auxiliary substances such as wetting or emulsifying agents, pH buffering agents, and/or compounds which enhance the effectiveness of the composition. The effectiveness of an auxiliary substances may be determined by measuring the induction of antibodies directed against a fusion cell.

The mammal to which the composition is administered is preferably a human, but can also be a non-human animal including but not limited to cows, horses, sheep, pigs, fowl (e.g., chickens), goats, cats, dogs, hamsters, mice and rats.

5.8 Effective Dose

The compositions of the present invention can be administered to a patient at therapeutically effective doses to prevent cancer. A therapeutically effective amount refers to that amount of the fusion cells sufficient to prevent or ameliorate the symptoms of such a disease or disorder, such as, e.g., regression of a pre-cancerous lesion or prevention of formation of such lesions in a person, particularly an individual at risk of developing cancer. Effective doses (immunizing amounts) of the compositions of the invention may also be extrapolated from dose-response curves derived from animal model test systems. The precise dose of fusion cells to be employed in the composition formulation will also depend on the particular type of disorder being prevented. For example, if a tumor is to be prevented from developing, the aggressiveness of the tumor is an important consideration when considering dosage. Other important considerations are the route of administration, and the nature of the patient. Thus the precise dosage should be decided according to the judgment of the practitioner and each patient's circumstances, e.g., the immune status of the patient, according to standard clinical techniques.

In a preferred embodiment, for example, to prevent formation of a human tumor, a fusion cell or fusion cell-cytokine composition, comprising non-dendritic pre-cancerous cells of the patient fused to dendritic cells are administered at a site away from the pre-cancerous lesion, preferably near lymph tissue. The administration of the composition may be repeated after an appropriate interval, e.g., every 3-6 months, using approximately 1×10⁸ cells per administration.

The present invention thus provides a method of immunizing a mammal, and preventing or treating development of a pre-cancerous lesion development or progression thereof in a mammal, comprising administering to the mammal a therapeutically effective amount of a fusion cell or a fusion cell-cytokine composition of the present invention.

5.9 Kits

The invention further provides kits for facilitating delivery of the immunotherapeutic composition according to the methods of the invention. The kits described herein may be conveniently used, e.g., in clinical settings to treat patients exhibiting symptoms of cancer or at risk of developing cancer. In one embodiment, for example, a kit is provided comprising, in one or more containers: a) a sample of a population of dendritic cells and b) instructions for its use in a method for treating or protecting against cancer or an infectious disease. An ampoule of sterile diluent can be provided so that the ingredients may be mixed prior to administration. In another embodiment the kit further comprises a cuvette suitable for electrofusion. In one embodiment, the dendritic cells are cryopreserved. In a further embodiment, the kit comprises a molecule that stimulates a humoral immune response and/or a cytotoxic T cell response. In a more preferred embodiment the stimulatory molecule is a cytokine such as, but not limited to interleukin-12.

### 6. EXAMPLE

### Prevention of Tumor Development by Vaccination with Fusion Cells

The present example demonstrates the prophylactic and therapeutic use of fusion cells formed by fusion of dendritic cells fused to non-dendritic cells that carry a specific allele at codon 1309 of the adenomatous polyposis coli (APC) gene that predisposes the host organism to colorectal cancer. Such APC1309 mice, which carry a mutation in codon 1309 of the APC gene, develop numerous gastrointestinal tumors. Vaccination of six-week-old APC1309 mice with fusion cells formed between dendritic cells and non-dentritic cells derived from tumors from an established APC1309 cell line inhibited the development tumors in the ileum. That is, the number of tumors present at ten weeks of age for treated mice was lower than that for untreated control mice, although both carried more tumors than baseline six-week-old APC1309 mice. However, administration of IL-12, as well as vaccination of six-week-old APC 1309 mice with fusion cells formed between dendritic cells and tumor cells from an established APC1309 cell line, not only inhibited the development of tumors in the ileum, but also resulted in the regression of pre-existing tumors. That is, the number of tumors present at ten weeks of age in mice vaccinated with fusion cells and also administered IL-12 was lower than that observed in baseline, six-week-old APC1309 mice.

Accordingly, these data support the prophylactic as well as the therapeutic efficacy of fusion cell vaccines comprising dendritic cells fused to non-dendritic cells carrying a specific allele that predisposes the host organism to cancer. More particularly, the data demonstrate the enhanced therapeutic efficacy of fusion cell vaccines which further comprise an immunostimulatory molecule, such as IL-12. Finally, although the non-dendritic cells in the mouse model used in the present example were generated from tumor cells, the techniques described here may be applied to, and thus serve as a model for, the isolation of pre-cancerous non-dendritic cells, and their use to generate fusions for use in prophylactic and therapeutic vaccines against cancer.

6.1 Materials and Methods

Mice

C57BL/6 mice were purchased from Sankyo Bio Laboratory (Tokyo). APC1309 knockout mice that have the genetic background C57BL/6 and have a mutation at codon 1309 of the APC gene, were provided by the Cancer Institute Tokyo, Japan (Quesada et al., Piroxicam and Acarbose as Chemopreventive Agents for Spontaneous Intestinal Adenomas in APC Gene 1309 Knockout Mice, Jpn. J Cancer Res. 89:392-396 (1998)). All of the experimental procedures were carried out in accordance with Jikei University guideline on animal welfare.

Before APC mice were used for experiments, their APC genotype was determined. DNA extracted from a tail sample of a four-week old mouse was collected by centrifugation, resuspended and mixed with 10-fold diluted PCR buffer (Takaya, Kyoto), 2.5 mM dNTPs (Takara, Kyoto), Taq polymerase (Takara, Kyoto), and distilled water. PCR was performed with 1 μl of first stand cDNA primer in a thermal cycler (Gene Amp PCR system 2400; Perkin Elmer, Shelton, Conn.). The primer was provided by the Cancer Institute, Tokyo. This primer was mixed with

APC27: 5′-TCAAGGTGCAGTTCATTATCATCACTG-3′;

APC47: 5′-CTTCAGTTGCAGGATCTTCAGCTGACC-3′; and

PGK-1: 5′-GCTAAAGCGCATGCTCCAGACTGCCTTG-3′. PCR products were separated using 2% agarose gel (Gibco, Grand Island, N.Y.), and detected by UV transillumination after ethidium bromide (Sigma, St. Louis, Mo.) staining. PCR products from wild-type C57BL/6 were 153 bp and those from APC1309 mice were 153 bp and 243 bp. The mice in which the analysis of PCR products showed both 153 bp and 243 bp bands were selected and used for the experiments.

Preparation of Dendritic Cells and Fusion Cells and Fusion with Non-Dendritic Cells

Dendritic cells were prepared by the method described by Inaba et al. (Inaba et al, 1993, J Exp Med 176, 1693-1702; Inaba et al., 1993, Proc Natl Acad Sci USA 90, 3038-3042). A cell line designated as T-tumor (or APC1309 tumor), established from the intestinal tumor of APC1309 mice, was provided by the Cancer Institute, Tokyo (Quesada et al., 1998, Jpn. J Cancer Res. 89:392-396). Dendritic cells were fused with trypsinized APC1309 tumor cells according to Gong et al. (Gong et al., 1997, Nat Med 3, 558-561).

More specifically, dendritic cells were isolated from bone marrow flushed from long bones of APC 1309 mice, and red cells were lysed with ammonium chloride (Sigma, St. Louis, Mo.). Lymphocytes, granulocytes and T cells were depleted from the bone marrow cells and the cells were plated in 24-well culture plates (1×10⁶ cells/well) in RPMI 1640 medium supplemented with 5% heat-inactivated FBS, 50 μM 2-mercaptoethanol, 2 mM glutamate, 100 U/ml penicillin, 100 pg/ml streptomycin, 10 ng/ml recombinant murine granulocyte-macrophage colony stimulating factor (GM-CSF; Becton Dickinson, San Jose, Calif.) and 30 U/ml recombinant mouse interleukin-4 (IL-4; Becton Dickinson). On day 5 of culture, nonadherent and loosely adherent cells were collected and replated on 100-mm petri dishes (1×10⁶ cells/mi; 10 ml/dish). GM-CSF and IL-4 in RPMI medium were added to the cells and 1×10⁶ Dendritic cells were mixed with 3×10⁶ irradiated (50 Gy, Hitachi MBR-1520R, dose rate: 1.1 Gy/min) APC1309 cells. After 48 hours, fusion was started by adding dropwise over 60 sec, 500 μl of a 50% solution of polyethylene glycol (PEG 1500; Sigma, St. Louis, Mo.). The fusion was stopped by stepwise addition of 30 ml. of serum-free RPMI medium. Fusion cells were plated in 100-mm petri dishes in the presence of GM-CSF and IL-4 in RPMI medium for 48 hr.

Treatment of Mice and Enumeration of the Tumors

Fusion cells (2×10⁵/mouse) were injected into the tail vein of the subject mice at 6 and 8 weeks of age. IL-12 (500 μg/mouse) was administered intraperitoneally at 5 and 10 days after the first injection of fusion cells and once again at 9 weeks of age. In specific experiments, mice were treated with either fusion cells alone or IL-12 alone. Mice were sacrificed at 10 weeks of age and gastrointestinal tracts, which extended from the lower esophagus to the rectum, were excised, fixed by infusion of formaldehyde from the rectum, cut open and stained with methylene blue. The tumors in the whole gastrointestinal tract were counted under a 10-power dissecting-microscope. In some experiments, anti-asialo-GM1 antibody (100 μl of a 0.25 mg/ml solution per mouse) was administered intravenously to the mice two days before and again two days after vaccination with fusion cells in order to abolish NK cell activity.

Assay of Cytotoxicity of Splenocytes to APC 1309 Tumor Cells

Splenocytes were prepared by gentle disruption of spleen on a steel mesh and cultured in medium containing 50U/ml of human recombinant IL-2 for 4 days and then examined for cytotoxic activity against APC1309 tumor cells. In some experiments, they were cultured with fusion cells or irradiated APC1309 tumor cells in the medium without IL-2 for 4 days and then examined for cytotoxic activity. APC 1309 tumor target cells, (1×10⁴ cells/well), were labeled with ⁵¹Cr, washed and incubated with the splenocytes at effector : target ratios ranging from 20:1 to 80:1 at 37° C. for 4 hours in 200 μl of RPMI-1640 medium supplemented with 10% heat inactivated fusion cells. After the cells were spun down by centrifugation, 1001 μl of supernatant was collected for measurement of radioactivity. The percent specific ⁵¹Cr release was calculated according to the following formula: percent ⁵¹Cr release=100×(cpm experimental release-cpm spontaneous release)/(cpm maximum release-cpm spontaneous release). The maximum release was that obtained from target cells incubated with 0.33N HCl and spontaneous release was that obtained from target cells incubated without the effector cells.

Detection of Antibody Reactive with APC 1309 Tumor Cells

Blood was collected by cardiac puncture and serum was separated by centrifugation. A pellet of trypsinized APC1309 tumor cells (2×10⁵) was mixed with 50μl of diluted mouse serum and incubated at 4° C. for 30 minutes. The cells were then washed twice with phosphate-buffered saline (PBS) and then incubated with 2 μl of FITC-conjugated rat anti-mouse immunoglobulin polyclonal antibody, or goat anti-mouse IgG1, IgG2a, IgG2b or IgG3 antibody (PharMingen, San Diego) at 4° C. for 30 minutes under the shield light. The cells were washed twice with PBS and their fluorescent intensities were measured by flowcytometric analysis using FACS (Becton Dickinson Immunocytometry Systems). The antibody activity was represented by the median value of whole fluorescent intensity histogram.

Detection of the Serum Cytotoxicity

APC1309 tumor cells (1×10⁵) were incubated in the absence or the presence of sera from untreated mice and from mice administered fusion cells and IL-12, for 48 hours at 37° C. in a 24 well plate. The sera were added to give a final dilution of 300 fold. After incubation, the cells were washed, trypsinized and stained with Trypan Blue. The total number of unstained cells was determined with a hemocytometer. When heat-inactivated serum was used, the serum was inactivated by incubation at 56° C. for 30 minutes.

Statistics

Statistical analyses were done by Student's T-test, using StatView-J5.0 software.

6.2 Results

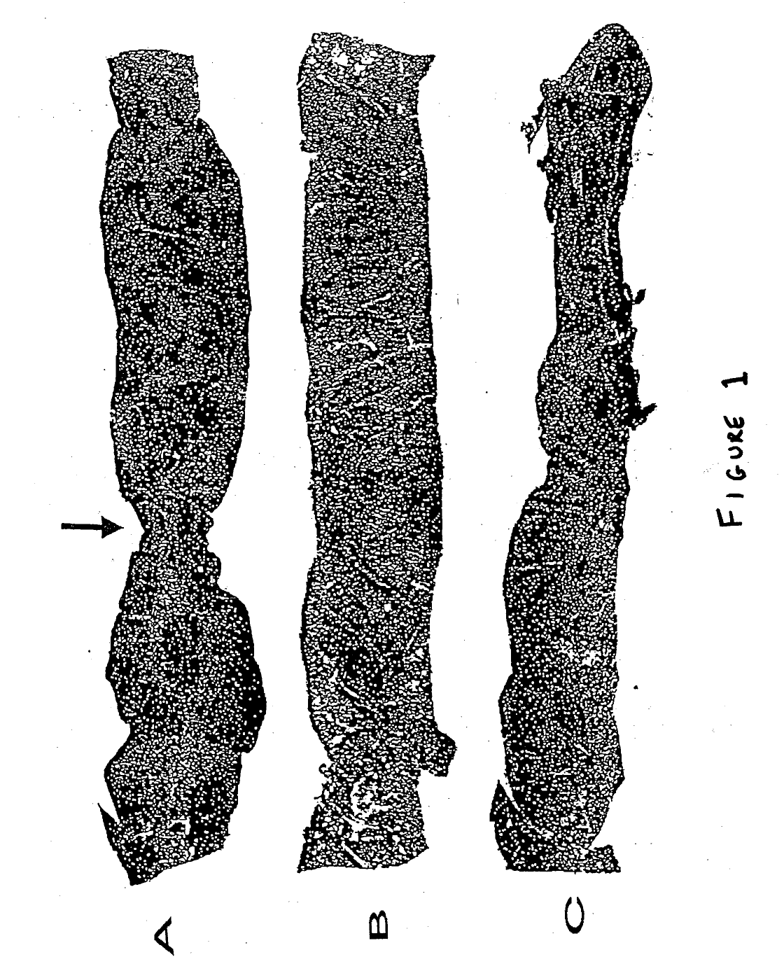

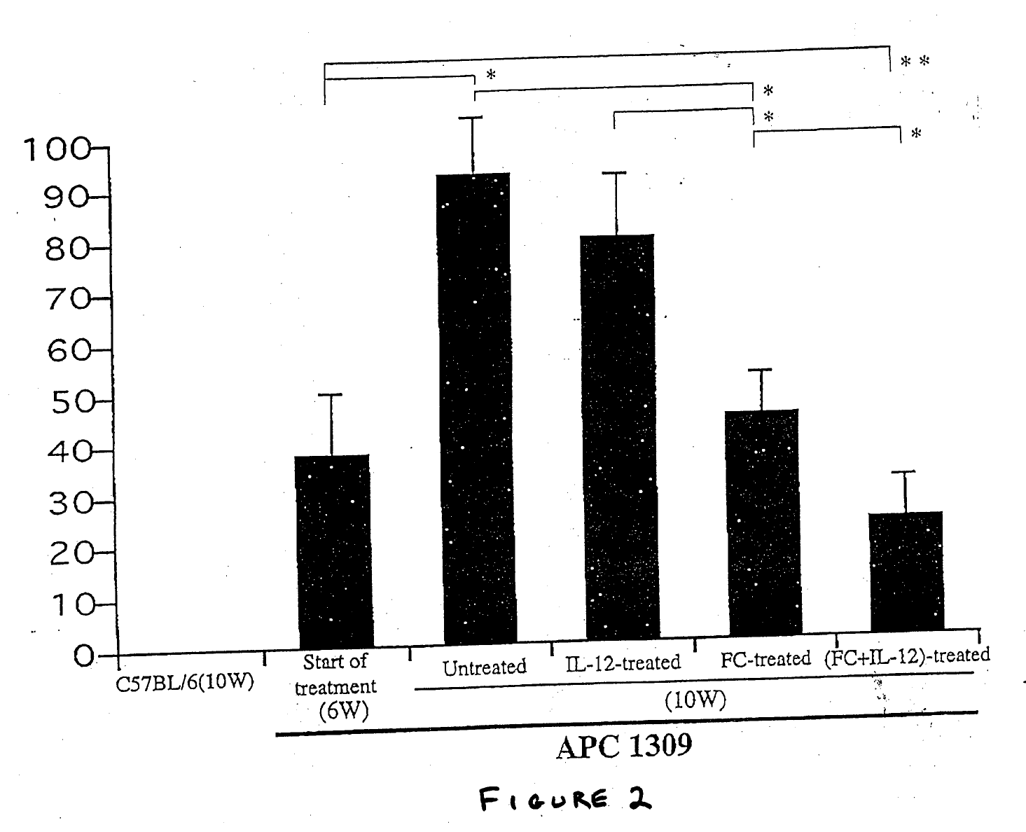

FIG. 1 shows macroscopic views of the small intestines of APC1309 mice. Fewer tumors were seen in the small intestines of mice treated with fusion cells comprising dendritic cells fused with APC1309 tumor cells of a cell line from an intestinal tumor of a APC mouse, than in the small intestine of an untreated mouse.

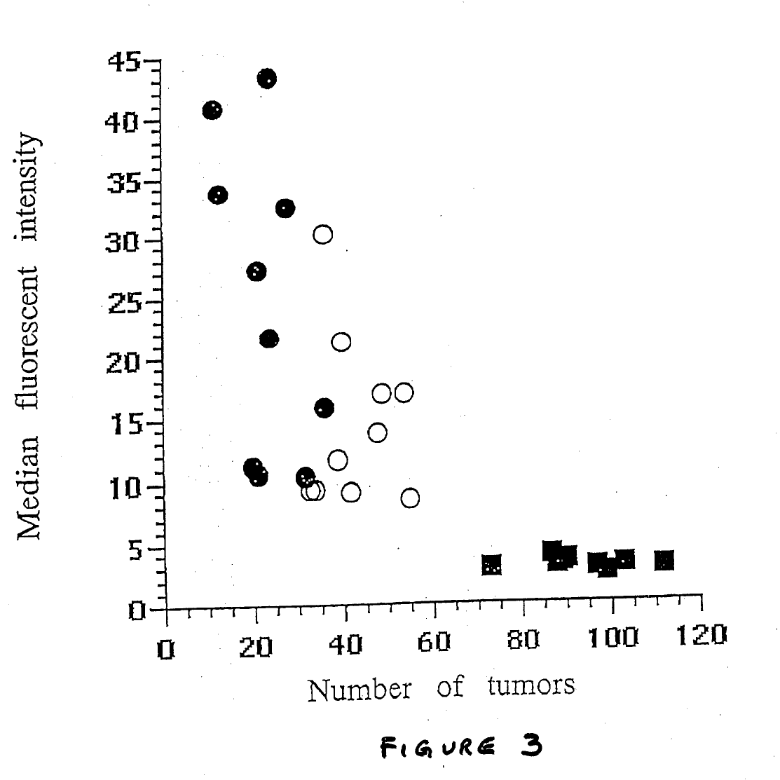

The mean number of gastrointestinal tumors was 38.0±11.7/mouse at 6 weeks of age and increased to 92.6±11.2/mouse at 10 weeks of age in untreated APC1309 mice (FIG. 2). In fusion-cell-treated mice, the mean tumor number was 44.1±8.0/mouse at 10 weeks of age, significantly lower than observed in the untreated mice (P<0.0001). Furthermore the mean number of tumors at 10 weeks of age in fusion cell-treated mice was not significantly different from that at 6 weeks of age when the treatment was started (P=0.2). When mice were administered fusion cells and IL-12, tumor development was further suppressed as compared with the mice treated with fusion cells alone (P<0.0001). Moreover the mean tumor number in fusion cells and IL-12-treated mice at 10 weeks of age was significantly smaller than in the mice at 6 weeks of age (P=0.003). Treatment with IL-12 alone did not elicit a significant antitumor effect. The inhibition of increase in the tumor number by the treatment with fusion cell was observed in all portions of gastrointestinal tract except the caecum where only a few tumors occurred. The size of the tumors ranged from 0.9 to 4.7mm in the long axis. The distribution of tumor size did not differ significantly among the groups of untreated, IL-12-treated, fusion cell-treated and fusion cells and IL-12-treated mice.

There were many enlarged lymphoid follicles on the mucosal surface of the intestines of fusion cell treated and fusion cell and IL-12-treated mice. However no inflammatory change was observed in non-tumorous region of the intestine.

Cytotoxic Activity of Splenocytes From Fusion Cell-Immunized Mice

Since freshly isolated splenocytes from fusion cell-treated and fusion cell and interleukin 12-treated mice showed no significant enhancement of cytotoxic activity against APC 1309 tumor cells in vitro, they were stimulated in vitro by various ways. No significant increase in the cytotoxic activity against APC 1309 tumor cells was demonstrated despite culture of the splenocytes in the presence of IL-2, irradiated tumor cells or fusion cell for 4 days. The absence of cytotoxic activity may be due to low expression of MHC molecules on the tumor cells. However pretreatment of tumor cells with IFN-γ, which is known to enhance expression of MHC class molecules did not make APC1309 tumor cells more susceptible to cytotoxic attack by splenocytes. When wild-type C57BL/6 mice were treated with fusion cells, the cytotoxic activity of their splenocytes was not induced either. NK cell activity of splenocytes from fusion cell-immunized mice was not enhanced judging from the results of cytotoxic assay using Yac-1 as the target cells. Furthermore administration of anti-asialo-GM1 antibody to fusion cell and interleukin 12-treated mice did not abolish antitumor activity.

Detection of Antibody to APC1309 Tumor Cells

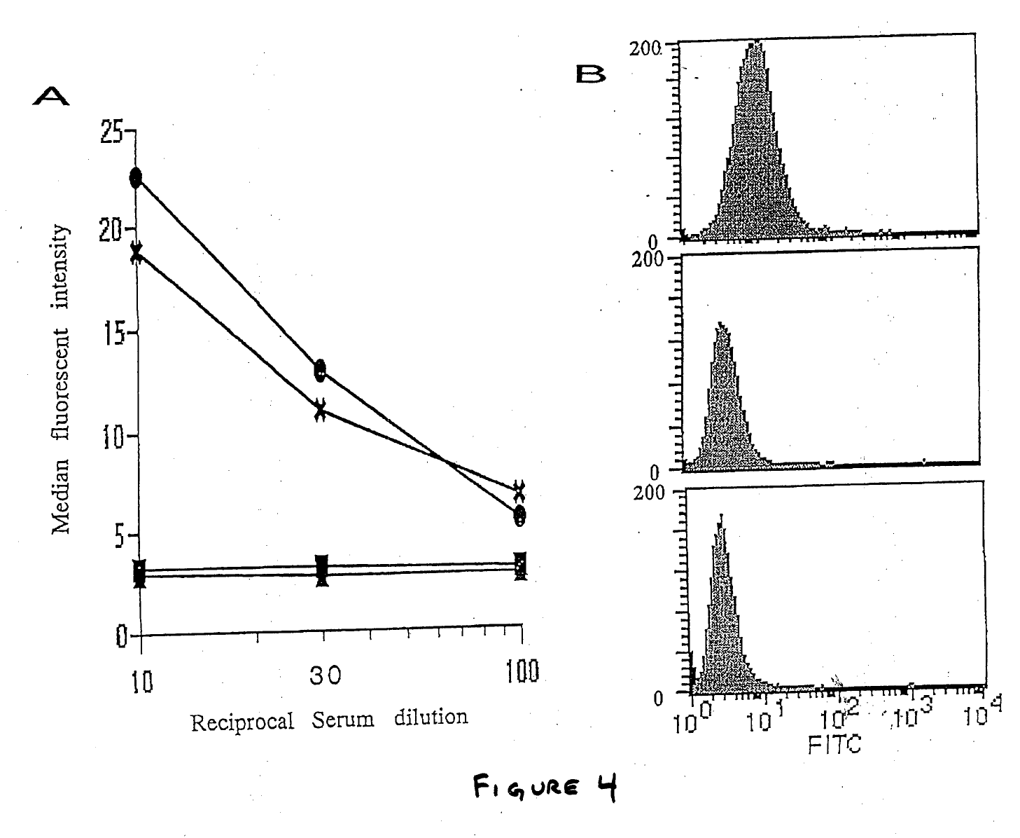

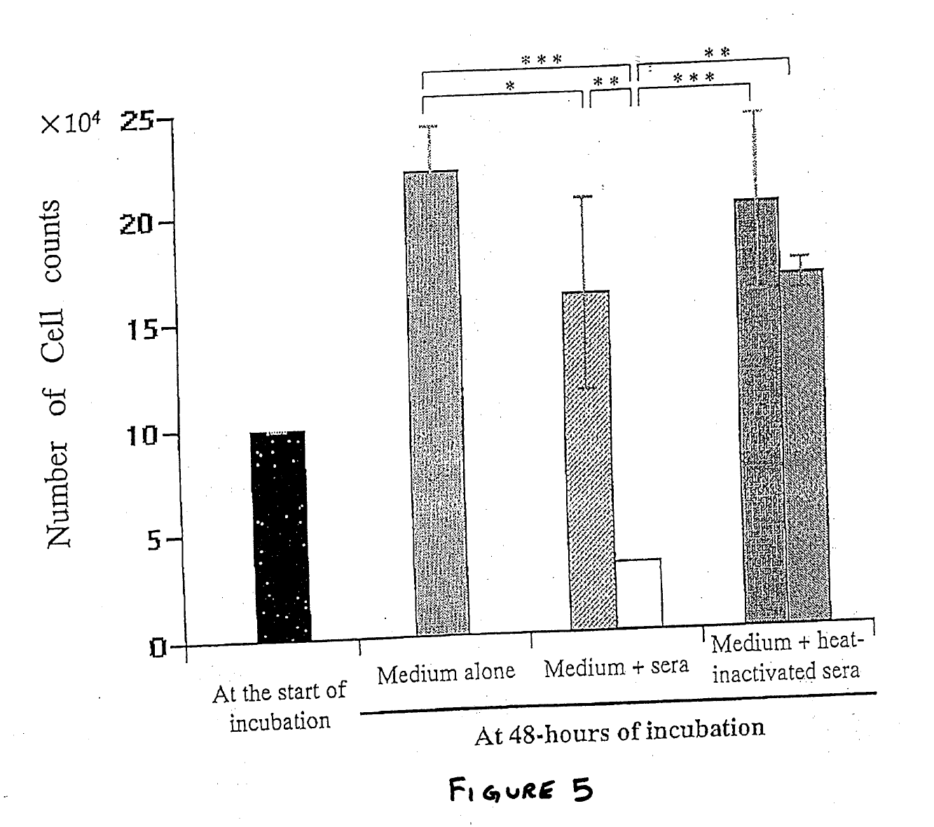

APC1309 tumor cells were incubated with sera from individual mice and then FITC-conjugated anti-mouse immunoglobulin was added. Fluorescent intensity of the labeled cells was measured by flowcytometric analysis using FACS (FIG. 3). The median intensities of fluorescent histograms shown by the tumor cells incubated with the sera from mice treated with fusion cells or fusion cells and IL-12 were significantly higher than those shown by the tumor cells incubated with sera from untreated mice (P<0.001). Cells incubated with sera from fusion cell and IL-12-treated mice gave higher fluorescent intensity than those incubated with mice treated with fusion cells alone (P=0.032). As indicated in FIG. 3, an inverse correlation was noted between the median fluorescence intensity and the number of tumors at 10 weeks of age. The median intensity declined with dilution of sera (FIG. 4A). Incubation of serum from a fusion cell and interleukin-12-treated mouse with APC1309 tumor cells reduced the fluorescent intensity to that shown by the tumor cells incubated with the serum from an untreated mouse (FIG. 4B). The decrease in the median intensity was not observed when the serum from fusion cell and interleukin-12-treated mouse was incubated with MC38/MUC-1 colon carcinoma cells, B-16 melanoma cells and Hepa-16 hepatocellular carcinoma cells (data not shown). The immunoglobulin isotype analysis with IgG1-, IgG2a-, IgG2b- and IgG3-specific antibodies revealed that the immunoglobulin isotype associated with the APC1309 tumor cells was IgG1. These findings indicated that sera from fusion cell treated or fusion cell and interleukin-12-treated mice had IgG1 class antibody that recognized APC1309 tumor cells. The antibody activity was represented by the median value of fluorescent intensity histogram. Antibody against APC 1309 tumor cells was also produced by inoculation of a parental C57BL/6 mouse with fusion cells (data not shown).

The Effect of Serum from Fusion Cell and Interleukin 12-treated Mice on APC1309 Tumor Cells in Vitro

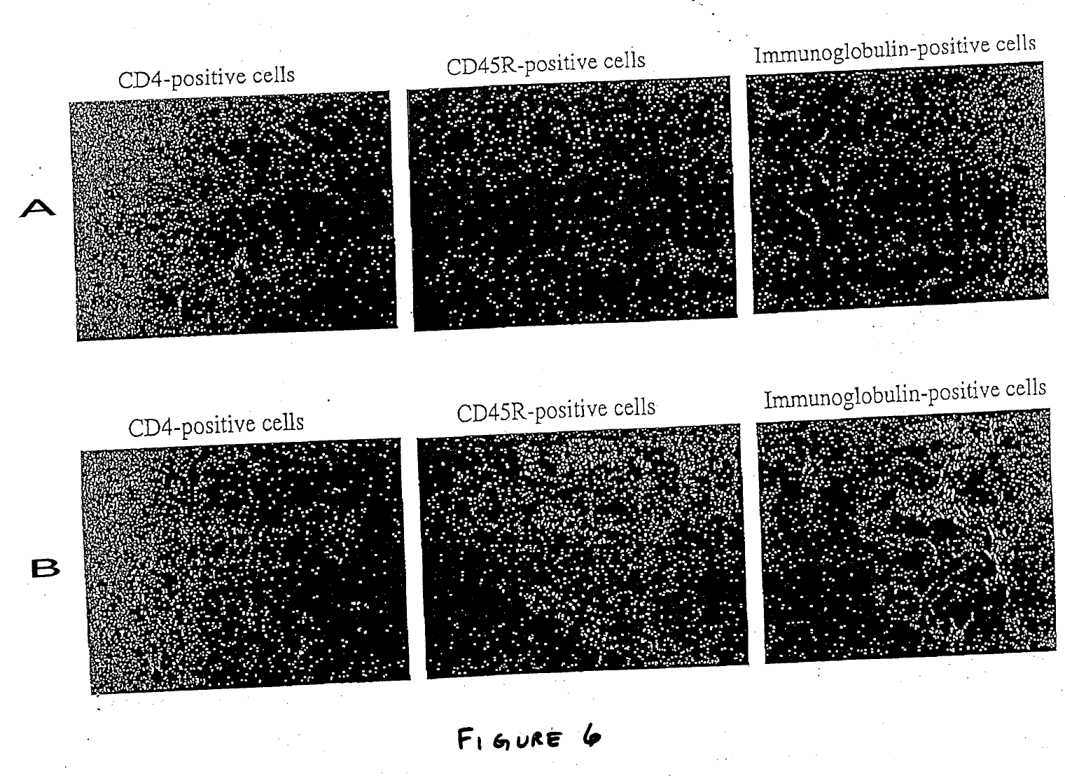

APC 1309 tumor cells were cultured in the presence of 300-fold diluted sera from untreated mice and from mice treated with fusion cells and interleukin 12 for 48 hours. The presence of serum from an untreated mouse decreased the viable cells (P<0.05). However the number of viable tumor cells after culture in the presence of serum from a (fusion cell and IL-12)-treated mouse was significantly smaller than that after the serum from an untreated (P<0.0001) (FIG. 5). Heat treatment of sera obscured the difference of the number of tumor cells that survived.

Immunohistochemical Study on Tumor Tissue in Untreated or Fusion Cell and Interleukin-12-treated Mice

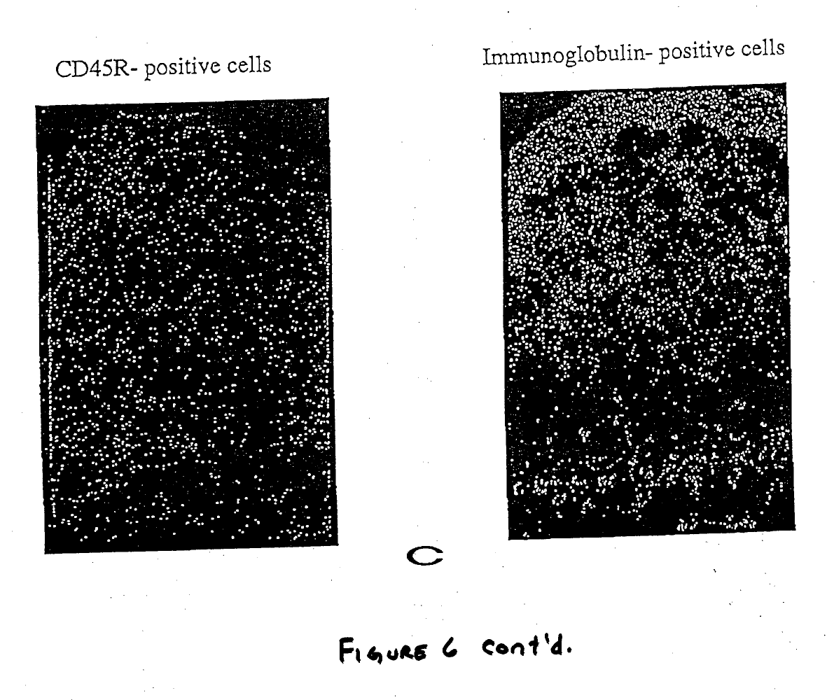

Very few CD4⁺, CD45R⁺ and immunoglobulin-positive cells were observed in the tumor tissue of untreated mice. In contrast, abundant CD45R⁺ and immunoglobulin-positive cells infiltrated tumor tissues of fusion cell and interleukin-12-treated mice (FIG. 6A). But CD4⁺ T-cells were observed in the tumor tissue of fusion cell and interleukin 12-treated mice, not the untreated mouse; few CD8⁺ T-cells were seen in the tumor tissue of fusion cell and interleukin 12-treated mice or in the tumor tissue of untreated mice.

Many lymphoid follicles were observed and extensive infiltration of CD45R⁺ and immunoglobulin-positive cells were detected on the intestinal mucosal surface of fusion cell-treated mice, but not in untreated mice (FIG. 6B).

6.3 Discussion

In this study, intravenous administration of dendritic cells fused with APC1309 tumor cells of an established cell line from the colon cancer of APC1309 mice, prevented an increase in tumor number. In an APC1309 untreated mouse, about 100 tumors developed at 10 weeks of age in the whole gastrointestinal tract. Fusion cell-treatment decreased the number of tumors to one half of that in the untreated controls. Treatment with fusion cell in combination with interleukin-12 brought about a further reduction in the number of tumors observed. In fusion cell and interleukin-12-treated mice, the number of tumors was significantly lower at 10 weeks of age than at 6 weeks of age. Antitumor activity of interleukin-12 was reported by Brunda (Brunda et al., 2000, J Exp Med 178, 1223-1230) and Nastala (Nastala et al., 1994, J Immunol 153, 1697-1706). However the treatment of mice with interleukin-12 alone did not suppress the increase in the number of tumors significantly in the present study, suggesting that interleukin-12 enhances antitumor immunity induced by the treatment with fusion cells as discussed below.

It has been reported that CTL are the effector cells in antitumor immunity induced by dendritic cells loaded with tumor antigens (Paglia et al., 1996, J Exp Med 183: 317-322; Mayordomo et al., 1996, Nature Med 1(12), 1297-1302; Butterfield et al., 1998, J Immunol 161: 5607-13; Condon et al.,. 1996, Nature Medicine 2:, 1122-1128; Gong et al., 1997, Nat Med 3: 558-561. In the present study, however, dendritic cells fused with APC1309 tumor cells failed to enhance cytotoxic activity of splenocytes against APC1309 tumor cells, or NK cell activity against APC1309 tumor cells. However, antibodies directed against APC1309 tumor cells were detected in the sera of fusion cell-treated mice. The inverse correlation between the number of tumors observed and the antibody activity detected indicates that antitumor immunity was effected by antibodies to APC1309 tumor cells. Supporting the involvement of antibody in the antitumor immunity, incubation of tumor cells in the presence of serum positive for serum antibodies reduced tumor-cell viability. This cytotoxic effect of serum was abolished by heat-treatment of the serum, suggesting that the serum cytotoxicity was complement-mediated. Participation of humoral immunity in the antitumor immunity was also substantiated by immunohistochemical studies. Only a few CD8⁺ T-lymphocytes were seen in the tumor tissue of APC1309 mice treated with fusion cells, but abundant CD45R⁺ and immunoglobulin-positive cells were observed. Furthermore there were many enlarged lymphoid follicles which were densely infiltrated with CD45R⁺ cells on the intestinal mucosal surface of fusion cell and interleukin-12-treated mice, presumably forming germinal centers for differentiation, maturation and proliferation of B cells producing the antitumor antibodies.

Dendritic cells play a critical role in the differentiation of naive T cells into TH₁ or TH₂ subsets (Macatonia et al., 1993, Int. Immunol 5:1119-28; Hilkens et aL, 1997, Blood 90:1920-1926; Ronchese et al., 1994, Eur. J Immunol. 24:1148-1154; Stumbles et al., 1998, J Exp. Med. 188: 2019-2031. Polarization of TH response is depends on the level of interleukin-12 produced by dendritic cells (Hilkens et al., 1997 Blood 90:1920-1926; Ronchese et al., 1994, Eur. J Immunol. 24:1148-1154; Stumbles et al., 1998, J. Exp. Med. 188: 2019-2031; Snijders et al., 1998, Int. Immunol. 10:1593-1598). Interleukin-12 induces a Th₁-polarized response promoting cell-mediated immunity. As discussed above, antitumor activity induced by treatment of APC1309 mice with fusion cells was mediated by the humoral antibody activity and was enhanced by administration of interleukin-12. Furthermore the immunoglobulin isotype of antitumor antibody detected was IgG 1, which characterized TH₂ response, in fusion cell and interleukin-12 treated mice. The enhancement of antitumor immunity by administration of interleukin-12 is surprising, because this cytokine can suppress a TH₂ response. However, interleukin-12 can enhance or inhibit humoral immunity, depending on immunoglobulin isotype and the stimulus to antibody formation. It was reported that the isotype of antibody immunoglobulin induced by the administration of interleukin-12 to mice was IgG2a, IgG2b and IgG3, whereas IgG1 antibody response was suppressed by this cytokine (Buchanan et al., 1995, Int. Immunol. 7:1519-1528). Once an IgG1 antibody response had been primed and boosted several times, however, it was enhanced by IL-12, though modestly (Buchanan et al., 1995, supra). In the present study, fusion cells were administered twice and the tumors existed continuously in the gastrointestinal tract, providing the stimulus to the immune system and presumably leading to the enhanced IgG1 antibody response.

There had been several reports of successful induction of antitumor immunity by immunization with dendritic cells loaded with tumor antigens in experimental animals (Paglia et al., 1996, J Exp Med 183: 317-322; Mayordomo et al., 1996, Nature Med 1(12): 1297-1302; Butterfield et al., 1998, J Immunol 161: 5607-13; Condon et al., 1996, Nature Medicine 2: 1122-1128; Gong et al., 1997, Nat Med 3: 558-561). However, in these studies, the antitumor immunity induced was reported to be cell-mediated, in contrast to the present data demonstrating antitumor immunity based on induction of antibody production. Production of interleukin-12 by dendritic cells was modulated by pathogen and the microenvironment including locally-secreted cytokines and inflammatory mediators (Kalinski et al., 1992, Immunology Today 20:561-567. Interleukin-12-producing capacity of dendritic cells was also modulated by their localization. Dendritic cells in the gut and the airway mucosa are more efficient inducers of TH₂ response than splenic dendritic cells (Stumbles et al., 1988, J Exp. Med. 188: 2019-2031; Snijders et al., 1998, Int. Immunol. 10:1593-1598; Buchanan et al., 1995, Int. Immunol. 7: 1519-1528; Kalinski et al., 1999, Amr. J Physiology 276: 1074-1078). Formation of germinal centers in the intestinal mucosa suggests that the gut lymphoid system participated in induction of antitumor immunity demonstrated in the present study, presumably resulting in the polarized, TH₂ response.

The present results demonstrate that immunization with dendritic cells fused with gastrointestinal tumor cells is useful for prevention of colon cancer development in FAP patients. Similarly, dendritic cells fused with gastrointestinal precancerous cells may also be used as vaccines for prevention of colon cancer development in FAP patients. This treatment is also useful in the prevention of recurrence of colon cancer in non-FAP patients, who have undergone surgical resection of the cancer.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated by reference herein in their entireties for all purposes.

What is claimed is:

1. A method of preventing cancer in a mammal, said method comprising administering to a mammal in need of said prevention an effective amount of fusion cells, wherein each said fusion cell is formed by the fusion of a dendritic cell and a non-dendritic cell and shares at least one MHC class I allele with said mammal, and wherein said non-dendritic cell displays at least one antigen having the antigenicity of an antigen specific to said cancer.

2. The method of claim 1, wherein said non-dendritic cell is a pre-cancerous non-dendritic cell.

3. The method of claim 2, wherein said pre-cancerous non-dendritic cell is the same cell type as the cancer to be prevented.

4. The method of claim 1, wherein said cancer is selected from the group consisting of renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenstrom's macroglobulinemia, and heavy chain disease.

5. A method of treating a pre-cancerous lesion in a mammal, said method comprising administering to a mammal in need of said treatment a therapeutically effective amount of fusion cells, wherein each said fusion cell is formed by the fusion of a dendritic cell and a pre-cancerous non-dendritic cell and shares at least one MHC class I allele with said mammal, and wherein said pre-cancerous non-dendritic cell displays at least one antigen having the antigenicity of an antigen specific to said pre-cancerous lesion.

6. The method of claim 5, wherein said pre-cancerous non-dendritic cell is the same cell type as said pre-cancerous lesion cell type.

7. The method of claim 5, wherein said pre-cancerous non-dendritic cell is isolated from said pre-cancerous lesion.

8. The method of claim 5, wherein said pre-cancerous lesion is a precursor of a cancer selected from the group consisting of renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenström's macroglobulinemia, and heavy chain disease.

9. The method of claim 4 or 8, wherein the cancer is a adenocarcinoma.

10. The method of claim 4 or 8, wherein the cancer is a hepatoma.

11. The method of claim 1 or 5, wherein the pre-cancerous non-dendritic cell is a cell from a gastrointestinal polyp.

12. The method of claim 1 or 5, wherein the pre-cancerous non-dendritic cell is a hepatoma cell.

13. The method of claim 1 or 5, further comprising administration of a molecule that stimulates a humoral immune response or a cytotoxic T cell immune response.

14. The method of claim 13, wherein said molecule is a cytokine.

15. The method of claim 14, wherein the cytokine is interleukin-12.

16. The method of claim 1 or 5, wherein the dendritic cell is obtained from human blood monocytes.

17. The method of claim 1 or 5, wherein said pre-cancerous non-dendritic cell is obtained from a primary culture of pre-cancerous cells derived from said mammal.

18. The method of claim 1 or 5, wherein said dendritic cells are autologous to said mammal.

19. The method of claim 1 or 5, wherein said pre-cancerous non-dendritic cells are autologous to the mammal.

20. The method of claim 1 or 5, wherein said dendritic cells are allogeneic to the mammal.

21. The method of claim 1 or 5, wherein said pre-cancerous non-dendritic cells are allogeneic to the mammal.

22. The method of claim 1 or 5, wherein both said dendritic cells and said pre-cancerous are autologous to said mammal, said method further comprising administering a molecule that stimulates a humoral immune response or a cytotoxic T cell immune response.

23. The method of claim 22, wherein said molecule is a cytokine.

24. The method of claim 23, wherein the cytokine is IL-12.

25. The method of claim 1 or 5, wherein said dendritic cells are allogeneic to the mammal and wherein said pre-cancerous non-dendritic cells have the same class I MHC haplotype as the mammal.

26. The method of claim 1 or 5, wherein said pre-cancerous non-dendritic cells are recombinant cells transformed with a nucleic acid encoding an antigen that displays the antigenicity of a tumor-specific antigen.

27. The method of claim 1 or 5, wherein said mammal is a human.

28. The method of claim 1 or 5, wherein said mammal is selected from the group consisting of a cow, a horse, a sheep, a pig, a fowl, a goat, a cat, a dog, a hamster, a mouse and a rat.

29. A method for fusing human dendritic cells and pre-cancerous non-dendritic human cells comprising subjecting a population of dendritic cells and a population of pre-cancerous non-dendritic cells to conditions that promote cell fusion.

30. The method of claim 29, wherein said pre-cancerous non-dendritic cells are autologous to said dendritic cells.

31. The method of claim 29, wherein said cell fusion is accomplished by electrofusion.

32. The method of claim 29, further comprising the step of inactivating the population of fusion cells.

33. The method of claim 32, wherein said inactivating the population of fusion cells is accomplished by y irradiating said cells.

34. A kit comprising, in one or more containers, a population of dendritic cells and instructions for fusing said dendritic cells with pre-cancerous non-dendritic cells for administration to a mammal in need thereof.

35. The kit of claim 34, further comprising a cuvette suitable for electrofusion.

36. The kit of claim 34, wherein said dendritic cells are cryopreserved.

37. The kit of claim 34, further comprising a molecule that stimulates an immune response selected from the group consisting of humor immune responses, cytotoxic T cell responses, and combinations thereof, and instructions for use of said kit for preventing or treating cancer.

38. The kit of claim 37, wherein said molecule is a cytokine.

39. The kit of claim 38, wherein said cytokine is IL-12.

40. A pharmaceutical composition comprising a fusion cell comprising a dendritic cell fused to a pre-cancerous non-dendritic cell.

41. The pharmaceutical composition of claim 40, wherein the dendritic cell is a human cell.

42. The pharmaceutical composition of claim 40, wherein the pre-cancerous non-dendritic cell is a human cell.

43. The pharmaceutical composition of claim 40, wherein the pre-cancerous non-dendritic cell is freshly isolated or obtained from a primary cell culture.

44. The pharmaceutical composition of claim 40, further comprising a molecule that stimulates an immune response selected from the group consisting of humor immune responses, cytotoxic T cell responses, and combinations thereof.

45. The pharmaceutical composition of claim 44, wherein said molecule is a cytokine.

46. The pharmaceutical composition of claim 44, wherein said molecule is IL-12.
